(19) United States
(12) Patent Application Publication
(10) Pub. No.: US 2024/0000860 A1
(43) Pub. Date: Jan. 4, 2024

(54) METHODS FOR TREATING AUTISM SPECTRUM DISORDER

(71) Applicants: Maude M. David, Corvallis, OR (US); Christine Tataru, Corvallis, OR (US); Alexandra Rose Phillips, Corvallis, OR (US); Shoko Kawana, Brisbane, CA (US); Xiaochen Yin, Brisbane, CA (US); Marie Peras, Brisbane, CA (US); Todd Z. DeSantis, Brisbane, CA (US); Brianna Chrisman, Stanford, CA (US); Dennis Wall, Stanford, CA (US)

(72) Inventors: Maude M. David, Corvallis, OR (US); Christine Tataru, Corvallis, OR (US); Alexandra Rose Phillips, Corvallis, OR (US); Shoko Kawana, Brisbane, CA (US); Xiaochen Yin, Brisbane, CA (US); Marie Peras, Brisbane, CA (US); Todd Z. DeSantis, Brisbane, CA (US); Brianna Chrisman, Stanford, CA (US); Dennis Wall, Stanford, CA (US)

(21) Appl. No.: 18/302,656

(22) Filed: Apr. 18, 2023

Related U.S. Application Data
(63) Continuation-in-part of application No. PCT/US2021/055698, filed on Oct. 19, 2021.
(60) Provisional application No. 63/093,763, filed on Oct. 19, 2020.

(51) Int. Cl. A61K 35/74; A61K 31/231; A61P 25/00

(52) U.S. Cl. CPC A61K 35/74; A61K 31/231; A61P 25/00

(57) ABSTRACT

Methods and compositions are provided herein for treating autism spectrum disorder in a subject, using one or more metabolites such as glutamate, malate, ursodeoxycholate, 5-dodecenoate, N-acetyl-L-glutamate, citrate, glycodeoxycholate, and carboxyethyl aminobutyric acid (CEGABA) and/or using one or more bacterial species such as bacterial species from the Streptococcaceae, Lachnospiraceae, Ruminococcaceae, Bacteroidaceae, Butyricicoccaceae, and/or Pasteurellaceae families; from the Streptococcus, Blautia, Haemophilus, Faecalibacterium, Bacteroides, Roseburia, Fusicatenibacter, Lachnospira, and/or Agathobaculum genera, or Blautia wexlerae, Bacteroides vulgatus, Bacteroides ovatus, Roseburia inulinivorans, Roseburia intestinalis, Fusicatenibacter saccharivorans, and/or Agathobaculum butyriciproducens.

### CROSS-REFERENCE TO RELATED APPLICATION

This application is a Continuation-in-Part of PCT Application No. PCT/US2021/055698 filed on Oct. 19, 2021, which in turn claims the benefit of and priority to U.S. Provisional Application No. 63/093,763 filed on Oct. 19, 2020; each of these prior applications is herein incorporated by reference in its entirety.

### STATEMENT OF FEDERALLY SPONSORED RESEARCH

This invention was made with government support under Grant no. DA042954, awarded by the National Institutes of Health. The government has certain rights in the invention.

### TECHNICAL FIELD

The present disclosure is related to metabolites, e.g., metabolites from bacterial species, and using metabolites for treating autism spectrum disorder in a subject.

### BACKGROUND

Autism spectrum disorder (ASD) is a neurodevelopmental condition characterized by social and behavioral impairments. In addition to neurological symptoms, ASD subjects frequently suffer from gastrointestinal abnormalities implying a possible link between the gut microbiome and ASD gastrointestinal pathophysiology. With animal models and human fecal microbiota transplant trials suggesting causal relationships, the impact of intestinal microbial metabolism on the gut-brain axis is gaining increased attention for drug discovery purposes. Small molecules produced or converted by the intestinal microbes are of particular interest as they could not only act locally in the intestine but also have the potential to pass the gut barrier and further the blood-brain barrier to directly modulate brain activity related to the disease phenotype.

### SUMMARY

Provided herein are methods for treating autism spectrum disorder in a subject including administering to the subject a composition including a therapeutically effective amount of two or more metabolites selected from the group consisting of: glutamate, malate, ursodeoxycholate, 5-dodecenoate, N-acetyl-L-glutamate, citrate, glycodeoxycholate, and carboxyethyl aminobutyric acid (CEGABA).

Also provided herein are methods treating autism spectrum disorder in a subject including (a) detecting a dysbiosis associated with autism spectrum disorder in a sample from the subject; and (b) administering to the subject a composition including one or more metabolites selected from the group consisting of: glutamate, malate, ursodeoxycholate, 5-dodecenoate, N-acetyl-L-glutamate, citrate, glycodeoxycholate, and carboxyethyl aminobutyric acid (CEGABA).

In some embodiments, the composition comprises two, three, four, or more metabolites. In some embodiments, the composition comprises a therapeutically effective amount of glutamate, malate, ursodeoxycholate, 5-dodecenoate, N-acetyl-L-glutamate, citrate, glycodeoxycholate, or carboxyethyl aminobutyric acid (CEGABA).

Also provided herein are methods for treating autism spectrum disorder in a subject including administering to the subject a composition comprising two or more bacterial species selected from the group consisting of: Bifidobacterium bifidum, Eggerthella lento, Eisenbergiella massilien, Prevotella copri, Romboutsia timonensis, Blautia wexlerae, Ruminiclostridium siraeum, Bacteroides intestinalis, Faecalicatena lactaris, Dialister invisus, and Ruminococcus callidus.

Also provided herein are methods treating autism spectrum disorder in a subject including (a) detecting a dysbiosis associated with autism spectrum disorder in a sample from the subject; and (b) administering to the subject a composition comprising two or more bacterial species selected from the group consisting of: Bifidobacterium bifidum, Eggerthella lento, Eisenbergiella massilien, Prevotella copri, Romboutsia timonensis, Blautia wexlerae, Ruminiclostridium siraeum, Bacteroides intestinalis, Faecalicatena lactaris, Dialister invisus, and Ruminococcus callidus.

Also provided herein are methods of treating autism spectrum disorder or modulating anxiety in a subject including administering to the subject a composition comprising two or more bacterial species of a bacterial family selected from the group consisting of: Streptococcaceae, Lachnospiraceae, Ruminococcaceae, Bacteroidaceae, Butyricicoccaceae, and Pasteurellaceae.

Also provided herein are of treating autism spectrum disorder or modulating anxiety in a subject including (a) detecting a dysbiosis associated with autism spectrum disorder in a sample from the subject; and (b) administering to the subject a composition comprising two or more bacterial species of a bacterial family selected from the group consisting of: Streptococcaceae, Lachnospiraceae, Ruminococcaceae, Bacteroidaceae, Butyricicoccaceae, and Pasteurellaceae.

In some embodiments, the two or more bacterial species are of a bacterial genera selected from the group consisting of: Streptococcus, Blautia, Haemophilus, Faecalibacterium, Bacteroides, Roseburia, Fusicatenibacter, Lachnospira, and Agathobaculum. In some embodiments, the two or more bacterial species are of a bacterial species selected from the group consisting of: Blautia wexlerae, Bacteroides vulgatus, Bacteroides ovatus, Roseburia inulinivorans, Roseburia intestinalis, Fusicatenibacter saccharivorans, and Agathobaculum butyriciproducens. In some embodiments, the two or more bacterial species has a 16S rRNA selected from the group consisting of: SEQ ID NO 1-13.

Any of methods provided herein can also include detecting a dysbiosis associated with autism spectrum disorder in a sample from the subject. In some embodiments, the sample is a fecal sample.

In some embodiments, detecting the dysbiosis associated with autism spectrum disorder includes determining bacterial gene expression in the sample from the subject. In some embodiments, detecting the dysbiosis associated with autism spectrum disorder includes determining bacterial composition in the sample from the subject.

In some embodiments, detecting the dysbiosis associated with autism spectrum disorder comprises determining that a bacterial species from the Pasteurellaceae family, Ruminococcaceae family, Bacteroidaceae family, Butyricicoccaceae family, Streptococcus genus, Blautia genus, Haemophilus genus, Faecalibacterium genus, Bacteroides genus, Roseburia genus, Fusicatenibacter genus, Lachnospira genus, Agathobaculum genus or a combination thereof that is depleted in the sample from the subject. In some embodiments, the bacterial species that is depleted in the sample from the subject is selected from the group consisting of: Blautia wexlerae, Bacteroides vulgatus, Bacteroides ovatus, Roseburia inulinivorans, Roseburia intestinalis, Fusicatenibacter saccharivorans, and Agathobaculum butyriciproducens.

In some embodiments, detecting the dysbiosis associated with autism spectrum disorder comprises determining that a bacterial species from the Bacteroidaceae family, Lachnospiraceae family, Oscillospiraceae family, Anaerovoraceae family, Erysipelotrichaceae family, Christensenellaceae family, Bacteroides genus, Blautia genus, Holdemania genus, Borkfalki genus, Anaerotignum genus, Faecalicatena genus, or a combination thereof is enriched in the sample from subject. In some embodiments, the bacterial species that is enriched in the sample from the subject is selected from the group consisting of: Bacteroides thetaiotaomicron, Borfalki ceftriaxensis, and Faecalicatena torques.

In some embodiments, the subject has severe autism. In some embodiments, severe autism is identified using the Mobile Autism Risk Assessment (MARA).

In some embodiments, the method comprises administering the composition to the subject once, twice, or three times per day. In some embodiments, the composition is formulated for oral administration, optionally as a tablet, a capsule, a powder, or a liquid. In some embodiments, one or more compositions described herein may be provided as nutritional supplements (also referred to as dietary supplements, food supplements, and/or nutraceuticals) and/or pharmaceutical drugs (e.g., drugs that have been approved by the U.S. Food and Drug Administration (FDA) and/or a foreign counterpart thereof to treat ASD and/or another disease, disorder, or condition).

Any of the methods provided herein can also include administering another treatment for autism spectrum disorder to the subject.

In some embodiments, the subject was previously identified as having autism spectrum disorder. In some of the embodiments, the subject is a human.

Also provided herein are compositions including two or more metabolites selected from the group consisting of: glutamate, malate, ursodeoxycholate, 5-dodecenoate, N-acetyl-L-glutamate, citrate, glycodeoxycholate, and carboxyethyl aminobutyric acid (CEGABA). In some embodiments, the composition comprises three, four, or more metabolites. In some embodiments, the composition comprises a therapeutically effective amount of glutamate, malate, ursodeoxycholate, 5-dodecenoate, N-acetyl-L-glutamate, citrate, glycodeoxycholate, carboxyethyl aminobutyric acid (CEGABA).

Also provided herein are compositions including two or more bacterial species selected from the group consisting of: Bifidobacterium bifidum, Eggerthella lento, Eisenbergiella massilien, Prevotella copri, Romboutsia timonensis, Blautia wexlerae, Ruminiclostridium siraeum, Bacteroides intestinalis, Faecalicatena lactaris, Dialister invisus, and Ruminococcus callidus.

Also provided herein are compositions including two or more bacterial species of a bacterial family selected from the group consisting of: Streptococcaceae, Lachnospiraceae, Ruminococcaceae, Bacteroidaceae, Butyricicoccaceae, and Pasteurellaceae. In some embodiments, the two or more bacterial species are of a bacterial genera selected from the group consisting of: Streptococcus, Blautia, Haemophilus, Faecalibacterium, Bacteroides, Roseburia, Fusicatenibacter, Lachnospira, and Agathobaculum. In some embodiments, the two or more bacterial species are of a bacterial species selected from the group consisting of: Blautia wexlerae, Bacteroides vulgatus, Bacteroides ovatus, Roseburia inulinivorans, Roseburia intestinalis, Fusicatenibacter saccharivorans, and Agathobaculum butyriciproducens.

In some embodiments, the composition is formulated for oral administration, optionally as a tablet, a capsule, a powder, or a liquid. In some embodiments, the composition is administered to a subject once, twice, or three times per day.

As used herein, the phrases an “effective amount” or a “therapeutically effective amount” of an active agent or ingredient, or pharmaceutically active agent or ingredient, refer to an amount of the pharmaceutically active agent sufficient enough to reduce or eliminate one or more symptoms of the disorder or to effect a cure upon administration. Effective amounts of the pharmaceutically active agent will vary with the kind of pharmaceutically active agent chosen, the particular condition or conditions being treated, the severity of the condition, the duration of the treatment, the specific components of the composition being used, and like factors.

An “effective amount” or a “therapeutically effective amount” of an active agent or ingredient, or pharmaceutically active agent or ingredient, can also refer to an amount of a combination of two or more active agents or a combination of an active agent and another treatment (e.g., behavioral therapy, psychological therapy, and educational therapy) sufficient to reduce or eliminate one or more symptoms of the disorder or in some cases, to effect a cure upon administration. For example, a “therapeutically effective amount” of an active agent can refer to an amount of a combination of active agents or a combination of an active agent and another treatment (e.g., behavioral therapy, psychological therapy, and educational therapy) when an additive or synergistic effect is observed with the combination compared to administration of the active agent(s) and/or treatment(s) of autism spectrum disorder alone.

As used herein, the phrase an “effective amount” of a bacterial species can refer to an amount of the bacterial species sufficient to reduce or eliminate one or more symptoms of the disorder or in some cases, to effect a cure upon administration. Effective amounts of a bacterial species will vary with the bacterial species chosen, the particular condition or conditions being treated, the severity of the condition, the duration of the treatment, the specific components of the composition being used, and like factors. An “effective amount” can also refer to an amount of a combination of two or more bacterial species or a combination of a bacterial species and another treatment and/or other adjunct therapy sufficient to reduce or eliminate one or more symptoms of the disorder or in some cases, to effect a cure upon administration. For example, an “effective amount” can refer to an amount of a combination of bacterial species or a combination of a bacterial species and another treatment (e.g., a therapeutic agent) when an additive or synergistic effect is observed with the combination compared to administration of the bacterial species and/or treatment(s) of autism spectrum disorder alone.

As used herein, “subject” or “patient” refers to any subject, particularly a mammalian subject such as a human, for whom diagnosis, prognosis, or therapy is desired.

As used herein, “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or improve a patient or subject's quality of life.

The term “preventing” as used herein means the prevention of the onset, recurrence, or spread, in whole or in part, of the disease or condition as described herein, or a symptom thereof.

The term “administration” or “administering” refers to a method of giving an amount of an active agent, or a composition thereof, a bacterial species, or a composition thereof, or a treatment of autism spectrum disorder and/or other adjunct therapy to a subject. The method of administration can vary depending on various factors, e.g., the components of the composition, the site of the disease, and the severity of the disease.

“Microbiome” refers to the collection of microorganisms and viruses and/or their genes from a given environment. For example, “microbiome” can refer to the collection of the microorganisms and viruses and/or their genes from the gastrointestinal tract of humans. “Microbiota” refers to the microorganisms in a specific environment.

“Dysbiosis” refers to a state of the microbiota or microbiome of the gut or other body area (e.g., mucosal or skin surfaces or any other microbiota niche) of a subject (i.e., the host) in which the diversity and/or function of the ecological network is disrupted, e.g., as compared to the state of the microbiota or microbiome of the gut or other body area in a control population. A control population can include individuals that meet one or more qualifications such as individuals that have not been diagnosed with a disease or disorder (e.g., the same disease or disorder as the subject); individuals that do not have a known genetic predisposition to a disease or disorder (e.g., the same disease or disorder as the subject); or individuals that do not have a known environmental predisposition to a disease or disorder (e.g., the same disease or disorder as the subject); or individuals that do not have a known predisposition that would prevent treatment of and/or recovery from a disease or disorder (e.g., the same disease or disorder as the subject). In some embodiments, the individuals in the control population meet one of the above control population qualifications. In some embodiments, the individuals in the control population meet two of the above control population qualifications. In some embodiments, the individuals in the control population meet three of the above control population qualifications. In some embodiments, the individuals in the control population meet four of the above control population qualifications. In some embodiments, the control population is homogenous with respect to at least one of the qualifications. Any disruption in the microbiota or microbiome of a subject (i.e., host) compared to the microbiota or microbiome of a control population can be considered a dysbiosis, even if such dysbiosis does not result in a detectable decrease in health of the subject. Dysbiosis in a subject may be unhealthy for the subject (e.g., result in a diseased state in the subject), it may be unhealthy for the subject under only certain conditions (e.g., result in diseased state under only certain conditions), or it may prevent the subject from becoming healthier (e.g., may prevent a subject from responding to treatment or recovering from a disease or disorder). Dysbiosis may be due to a decrease in diversity of the microbiota population composition (e.g., a depletion of one or more bacterial species, an overgrowth of one or more bacterial species, or a combination thereof), the overgrowth of one or more population of pathogens (e.g., a population of pathogenic bacteria) or pathobionts, the presence of and/or overgrowth of a symbiotic organism able to cause disease only when certain genetic and/or environmental conditions are present in a subject, or a shift to an ecological network that no longer provides a beneficial function to the host and therefore no longer promotes health.

As used herein the terms “microorganism” or “microbe” should be taken broadly. These terms are used interchangeably and include, but are not limited to, the two prokaryotic domains, Bacteria and Archaea, as well as eukaryotic fungi and protists. In some embodiments, the disclosure refers to a “bacterium” or a “microbe.” This characterization can refer to not only the identified taxonomic bacterial genera of the microbe, but also the identified taxonomic species, as well as the bacterial species. A “strain” can include descendants of a single isolation in pure culture that is usually made up of a succession of cultures ultimately derived from an initial single colony. In some embodiments, a strain includes an isolate or a group of isolates that can be distinguished from other isolates of the same genus and species by phenotypic characteristics, genotypic characteristics, or both.

The term “relative abundance” as used herein, is the number or percentage of a microbe present in the gastrointestinal tract or any other microbiota niche of a subject, such as the ocular, placental, lung, cutaneous, urogenital, or oral microbiota niches, relative to the number or percentage of total microbes present in the gastrointestinal tract or the other microbiota niche of the subject. The relative abundance may also be determined for particular types of microbes such as bacteria, fungi, viruses, and/or protozoa, relative to the total number or percentage of bacteria, fungi, viruses, and/or protozoa present. Relative abundance can be determined by a number of methods readily known to the ordinarily skilled artisan, including, but not limited to, array or microarray hybridization, sequencing, quantitative PCR, and culturing and performance of colony forming unit (cfu, CFU) assays or plaque forming unit (pfu, PFU) assays performed on a sample from the gastrointestinal tract or other microbiota niche.

As used herein, terms such as “isolate” and “isolated” in reference to a microbe, are intended to mean that a microbe has been separated from at least one of the materials with which it is associated in a particular environment (for example gastrointestinal fluid, gastrointestinal tissue, human digestive fluid, human digestive tissue, etc.). Accordingly, an “isolated microbe” does not exist in its naturally occurring environment. In some embodiments, an isolated microbe, e.g., a bacterial species, may exist as, for example, a biologically pure culture, or as spores (or other forms of the bacterial species) in association with a pharmaceutically acceptable excipient suitable for human administration. In some embodiments, more than one microbe can be isolated. For example, “isolated microbes” can refer to a mixture of two or more microbes that have been separated from at least one of the materials with which they are associated in a particular environment.

In some embodiments, the isolated microbes exist as isolated and biologically pure cultures. As used herein, the term “biologically pure” refers to a composition comprising a species or strains of a microbe, wherein the composition is substantially free from the material from which the microbe was isolated or produced and from other microbes (e.g., other species or strains and other microbes of a different taxonomic classification). In some embodiments, “biologically pure” can refer to a composition that comprises a strain of a bacterial species that is substantially free from the material from which the bacterial species was isolated or produced and from other microbes, e.g., other strains of the same bacterial species, other species of the same bacteria, and other bacteria and/or microbes of a different taxonomic classification). It will be appreciated by one of skill in the art, that an isolated and biologically pure culture of a particular microbe, denotes that said culture is substantially free (within scientific reason) of other living organisms and contains only the individual microbe in question. As used herein, “substantially free” means that a composition comprising a species or strain of a microbe is at least about 80%, about 85%, about 90%, about 91%, about 92%, about 93%, about 94%, about 95%, about 96%, about 97%, about 98%, about 99%, or more than about 99% free of the material from which the microbe was isolated or produced and from other microbes. In some embodiments, a biologically pure composition contains no other bacterial species in quantities that can be detected by typical bacteriological techniques.

As used herein, “probiotic” refers to a substantially pure microbe (i.e., a single isolate) or a mixture of microbes, and may also include any additional components that can be administered to a subject (e.g., a human), for restoring or altering the microbiota or microbiome in the subject. In some embodiments, a probiotic or microbial inoculant composition can be administered with an agent to allow the microbe(s) to survive the environment of the gastrointestinal tract, i.e., to resist low pH and/or to grow in the gastrointestinal environment. In some embodiments, a composition as described herein includes a probiotic.

As used herein, “prebiotic” refers to an agent that increases the number and/or activity of one or more microbes. Such microbes can include microbes for restoring or altering the microbiota or microbiome of a subject. Non-limiting examples of a prebiotic include a fructooligosaccharide (e.g., oligofructose, inulin, or an inulin-type fructan), a galactooligosaccharide, an amino acid, an alcohol. See, for example, Ramirez-Farias et al. (2008. Br. J Nutr. 4:1-10) and Pool-Zobel and Sauer (2007. J Nutr. 137:2580-2584).

As used herein, a “live biotherapeutic product” or “LBP” refers to a biological product that: 1) contains live organisms, such as bacteria, and 2) is applicable to the prevention, treatment, and/or cure of a disease or condition of a subject.

A “combination” of two or more bacteria, e.g., bacterial species, can refer to the physical co-existence of the bacteria, either in the same material or product. In some embodiments, a combination of two or more bacteria can include the temporal co-administration or co-localization of the two or more bacteria.

Generally, a bacterial species genomic sequence will contain multiple copies of 16S rRNA sequences. The 16S rRNA sequences can be used for making distinctions between genera, species and strains. For example, if one or more of the 16S rRNA sequences shares less than 97% sequence identity from a reference sequence, then the two organisms from which the sequences were obtained can be of different species or strains.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/ on the world wide web or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “combination therapy” as used herein refers to a dosing regimen of one or more active agents (e.g., a metabolite) and one or more other treatments of autism spectrum disorder during a period of time, wherein the active agent(s) and other treatment (e.g., behavioral therapy, psychological therapy, educational therapy, a prebiotic, a probiotic, or a combination thereof) are administered together or separately in a manner prescribed by a medical care taker or according to a regulatory agency. As can be appreciated in the art, a combination therapy can be administered to a patient for a period of time. In some embodiments, the period of time occurs following the administration of one or more of: a different bacterial species, a different treatment/therapeutic agent, and a different combination of treatments/therapeutic agents to the subject. In some embodiments, the period of time occurs before the administration of one or more of: a different active agent, a different treatment, and a different combination of treatments/therapeutic agents to the subject.

The term “fixed combination” means that one or more active agents as described herein, or a composition thereof, and at least one other treatment (e.g., a prebiotic, a probiotic, or a combination thereof), are both administered to a subject simultaneously in the form of a single composition or dosage.

The term “non-fixed combination” means that one or more active agents as described herein, or a composition thereof, and at least one other treatment (e.g., a prebiotic, a probiotic, or a combination thereof) are formulated as separate compositions or dosages such that they may be administered to a subject simultaneously or sequentially with variable intervening time limits. These also apply to cocktail therapies, e.g., the administration of three or more therapeutic agents.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

The details of one or more embodiments of the invention are set forth in the accompanying drawings and the description below. Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is an exemplary schematic of the analysis.

FIG. 2 is a table showing cohort information. 16S NGS: 16S rRNA amplicon sequencing; MTG: metagenomics; *MTT: metatranscriptomics NMR: Nuclear magnetic resonance; LC-MS: Liquid chromatography-mass spectrometry.

FIG. 3 is an exemplary schematic of the analysis.

FIG. 4 is a plot showing alpha diversity measure and ASD severity.

FIGS. 5A and 5B are plots showing relative abundance and ASD severity.

FIGS. 6A and 6B are plots showing relative abundance of Anaerotignum, Blautia, and Blautia wexlerae at three time points.

FIGS. 7A and 7B are plots showing measured intensity for Metabolites A and B.

FIGS. 8A and 8B are plots showing number of metabolites by class (FIG. 8A) and contribution type (FIG. 8B).

FIG. 9A is a plot showing pairwise correlation for Metabolite A.

FIG. 9B is a plot showing pairwise correlation for Metabolite B.

FIG. 10 is a volcano plot comparing the metabolites abundance in the most severe cases of ASD and their neurotypical siblings.

FIG. 11 is a plot showing mouse weights over time when eating food supplemented with various metabolites.

FIG. 12 is a plot showing the amount of supplemented food mice ate.

FIG. 13 is a schematic of an elevated plus maze.

FIG. 14 is a plot of the time (seconds) spent in the closed arms of an elevated plus maze of mice fed food supplemented with various metabolites. Control=no supplement compound; 5D=supplemented with 5-dodecenoate; GCD=supplemented with glycodeoxycholate; UDC=supplemented with ursodeoxycholate. Significance was determined with a Wilcox test.

FIG. 15 is a plot of the activity time (seconds) in closed arms of an elevated plus maze of mice fed food supplemented with various metabolites. Control=no supplement compound; 5D=supplemented with 5-dodecenoate; GCD=supplemented with glycodeoxycholate; UDC=supplemented with ursodeoxycholate. Significance was determined with a Wilcox test.

FIG. 16 is a figure of an exemplary three-chamber sociability test.

FIG. 17 is a plot of the total distance traveled each day by mice fed food supplemented with various metabolites. Control=no supplement compound; 5D=supplemented with 5-dodecenoate; GCD=supplemented with glycodeoxycholate; UDC=supplemented with ursodeoxycholate. Significance was determined with a Wilcox test.

FIG. 18 is a plot of the time mice spent in the center of the maze by mice fed food supplemented with various metabolites. Control=no supplement compound; 5D=supplemented with 5-dodecenoate; GCD=supplemented with glycodeoxycholate; UDC=supplemented with ursodeoxycholate. Significance was determined with a Wilcox test.

FIG. 19 is a plot of the total ambulatory time of mice fed food supplemented with various metabolites. Control=no supplement compound; 5D=supplemented with 5-dodecenoate; GCD=supplemented with glycodeoxycholate; UDC=supplemented with ursodeoxycholate. Significance was determined with a Wilcox test.

FIG. 20 is a plot of the ambulatory time in the center of the center of the maze of mice fed food supplemented with various metabolites. Control=no supplement compound; 5D=supplemented with 5-dodecenoate; GCD=supplemented with glycodeoxycholate; UDC=supplemented with ursodeoxycholate. Significance was determined with a Wilcox test.

FIG. 21 is a schematic of a three-chamber sociability test.

FIG. 22 is a plot of time spent with a novel mouse (‘new’) or a known mouse (‘old’) in a three-chamber sociability test. Control=no supplement compound; 5D=supplemented with 5-dodecenoate; GCD=supplemented with glycodeoxycholate; UDC=supplemented with ursodeoxycholate. Significance was determined with a Wilcox test.

FIG. 23 is a plot of the time spent with a novel mouse in a three-chamber sociability test. Control=no supplement compound; 5D=supplemented with 5-dodecenoate; GCD=supplemented with glycodeoxycholate; UDC=supplemented with ursodeoxycholate. Significance was determined with a Wilcox test.

FIG. 24 is a plot of the average distance traveled over time by mice fed food supplemented with various metabolites. Controlaverage=average distance traveled of mice fed control food with no supplement; FiveDodecenoateAvg=average distance traveled by mice fed food supplemented with 5-dodecenoate; GDC_Average=average distance traveled mice fed food supplemented with glycodeoxycholate; UDC_Average=average distance traveled mice fed food supplemented with ursodeoxycholate.

FIG. 25 is a schematic of the study design of Example 4.

FIG. 26 is a plot showing relative abundance counts of ASVs significantly associated with the ASD cohort in two independent contrast methods. ASVs taxonomic annotation of the 16S amplicon (at the families, genus, and species) and the corresponding relative abundance for the 11 taxa identified in at least two independent contrast methods (ANCOM and/or MetagenomeSeq and/or DESeq2) over the three time points.

FIGS. 27A-27D are plots showing performance and variable importance of binary phenotype classifiers using different subsets of data. FIG. 27A is a plot showing the performance of predictive models using metadata and ASVs. Grey lines show the relationship between folds in 7 fold cross-validation. FIG. 27B is a plot showing the predictive value of individual lifestyle variables. X axis represents change in Gini index upon removal of the variable. FIG. 27C is a plot showing performance of predictive models using only ASV inputs. The 11 biomarker set (Table 4) classifies with an average ROC AUC of 0.66, and adding additional associated taxa does not significantly increase performance. FIG. 27D is a plot showing the predictive value of ASVs and their taxonomic annotations.

FIGS. 28A-28C are plots showing correlations between changes in anxiety and log 2-fold changes in relative taxa abundance. FIG. 28A is a plot showing the change of ASVs abundance correlated with changes in anxiety score across the entire cohort. Positive/negative values on the x-axis signify increases/decreases in anxiety respectively between timepoints within an individual, and positive/negative values on the y-axis represent an increased/decreased log 2 fold change between the relative abundance of an ASV between timepoints within the same individual. R2 and p values represent results from a spearman correlation. FIG. 28B is a plot showing ASVs correlated with changes in anxiety scores across both cohorts, and still significant when considering the ASD cohort only. FIG. 28C is a plot showing ASVs that correlate negatively with anxiety in the ASD cohort also correlate with alpha diversity (Shannon evenness index) of samples.

FIGS. 29A-29N show taxonomic composition in the colons of C57BL/6, CNTNAP2, and CNTNAP2 mice supplemented with 5D. FIG. 29A shows Principal Coordinate analysis (PCoA) plot of Bray-Curtis distances between treatments. FIGS. 29B-29N show differential abundance tests of ASVs aggregated at the genus level for different bacteria families.

FIGS. 30A-30D show results indicating that lipidomic profiles in brain (FIG. 30A), plasma (FIG. 30B), liver (FIG. 30C), and stool (FIG. 30D) shifted from CNTNAP2 mutant toward the wild type mice C57BL/6 with 5D supplementation.

FIGS. 31A-31D show results indicating that triglyceride levels in brain (FIG. 31A), liver (FIG. 31B), plasma (FIG. 31C), and stool (FIG. 31D) were elevated in CNTNAP2 mice and reduced significantly by 5D supplementation.

FIG. 32 shows gene set enrichment analysis results on RNA-seq data from the frontal cortex region affected by 5D supplementation, wherein bar size depicts enrichment score normalized by gene set size, and color depicts fdr corrected p-value as determined by gene set enrichment analysis.

### SEQUENCE LISTING

The nucleic acid and amino acid sequences are shown using standard letter abbreviations as defined in 37 C.F.R. 1.822. Only one strand of each nucleic acid sequence is shown, but the complementary strand is understood as included by any reference to the displayed strand.

A Sequence Listing XML submitted under 37 C.F.R. § 1.831(a) in compliance with §§ 1.832 through 1.834, is submitted herewith as “Sequence.XML,” created on Apr. 18, 2023, 20,480 bytes, which is incorporated by reference herein. In the accompanying sequence listing:
SEQ ID NO: 1 is an exemplary nucleotide sequence from a 16S rRNA gene of Faecalicatena lactaris.
SEQ ID NO: 2 is an exemplary nucleotide sequence from a 16S rRNA gene of Dialister invisus.
SEQ ID NO: 3 is an exemplary nucleotide sequence from a 16S rRNA gene of Faecalibacterium spp.
SEQ ID NO: 4 is an exemplary nucleotide sequence from a 16S rRNA gene of Bacteroides vulgatus.
SEQ ID NO: 5 is an exemplary nucleotide sequence from a 16S rRNA gene of Bacteroides ovatus.
SEQ ID NO: 6 is an exemplary nucleotide sequence from a 16S rRNA gene of Roseburia inulinivorans.
SEQ ID NO: 7 is an exemplary nucleotide sequence from a 16S rRNA gene of Roseburia intestinalis.
SEQ ID NO: 8 is an exemplary nucleotide sequence from a 16S rRNA gene of Faecalicatena torques.
SEQ ID NO: 9 is an exemplary nucleotide sequence from a 16S rRNA gene of Fusicatenibacter saccharivorans.
SEQ ID NOs: 10 is an exemplary nucleotide sequence from a 16S rRNA gene of Lachnospira spp.
SEQ ID NO: 11 is an exemplary nucleotide sequence from a 16S rRNA gene of a Lachnospiraceae sp.
SEQ ID NOs: 12 and 13 are exemplary nucleotide sequences from a 16S rRNA gene of Agathobaculum butyriciproducens.
SEQ ID NOs: 14 and 15 are exemplary primers.

### DETAILED DESCRIPTION

This document provides compositions and methods for treating subjects having autism spectrum disorder (ASD), and compositions and methods for modulation comorbidities of autism spectrum disorder (e.g., anxiety) in a subject using one or more metabolites and/or one or more bacterial species. ASD is a complex neurodevelopmental brain disorder that can be characterized by behavioral symptoms including impairments in social communication and restricted/repetitive behavior. See, e.g., Eissa et al. Front Neurosci. 2018; 12: 304. The severity of symptom can vary widely, and they can also be compounded by significant comorbidities including intellectual disability, epilepsy, anxiety, sleep, and gastrointestinal disorders. See Cheroni et al. Mol Autism. 2020; 11: 69. Evidence has shown that children with ASD can have an abnormal composition of the gut microbiota (gut dysbiosis), which may lead to systemic inflammation and neuroinflammation of the central nervous system. See Inoue et al, 2019. J. Clin. Biochem. Nutr. 64, 217-223.

The methods provided herein can include administering to the subject a composition that includes a therapeutically effective amount of a metabolite. In some embodiments, the composition comprises a therapeutically effective amount of one or more (e.g., two or more, three or more, four or more, five or more, six or more, seven or more) metabolites selected from the group consisting of: glutamate, malate, ursodeoxycholate, 5-dodecenoate, N-acetyl-L-glutamate, citrate, glycodeoxycholate, and carboxyethyl aminobutyric acid (CEGABA).

In some embodiments, the composition includes a therapeutically effective amount of two or more metabolites selected from the group consisting of: glutamate, malate, ursodeoxycholate, 5-dodecenoate, N-acetyl-L-glutamate, citrate, glycodeoxycholate, and carboxyethyl aminobutyric acid (CEGABA) (e.g., any two, three, four, five, six, seven or all eight of the metabolites described herein). In some embodiments, the composition includes a therapeutically effective amount of three or more metabolites selected from the group consisting of: glutamate, malate, ursodeoxycholate, 5-dodecenoate, N-acetyl-L-glutamate, citrate, glycodeoxycholate, and carboxyethyl aminobutyric acid (CEGABA). In some embodiments, the composition includes a therapeutically effective amount of four or more metabolites selected from the group consisting of: glutamate, malate, ursodeoxycholate, 5-dodecenoate, N-acetyl-L-glutamate, citrate, glycodeoxycholate, and carboxyethyl aminobutyric acid (CEGABA).

In some embodiments, the composition comprises glutamate. In some embodiments, the composition comprises malate. In some embodiments, the composition comprises ursodeoxycholate. In some embodiments, the composition comprises 5-dodecenoate. In some embodiments, the composition comprises N-acetyl-L-glutamate. In some embodiments, the composition comprises citrate. In some embodiments, the composition comprises glycodeoxycholate. In some embodiments, the composition comprises CEGABA.

In some embodiments, the composition comprises a therapeutically effective amount of glutamate. In some embodiments, the composition comprises a therapeutically effective amount of malate. In some embodiments, the composition comprises a therapeutically effective amount of ursodeoxycholate. In some embodiments, the composition comprises a therapeutically effective amount of 5-dodecenoate. In some embodiments, the composition comprises a therapeutically effective amount of N-acetyl-L-glutamate. In some embodiments, the composition comprises a therapeutically effective amount of citrate. In some embodiments, the composition comprises a therapeutically effective amount of glycodeoxycholate. In some embodiments, the composition comprises a therapeutically effective amount of CEGABA.

In some embodiments, the composition comprises glutamate and one or more of malate, ursodeoxycholate, 5-dodecenoate, N-acetyl-L-glutamate, citrate, glycodeoxycholate, and carboxyethyl aminobutyric acid (CEGABA). In some embodiments, the composition comprises malate and one or more of glutamate, ursodeoxycholate, 5-dodecenoate, N-acetyl-L-glutamate, citrate, glycodeoxycholate, and carboxyethyl aminobutyric acid (CEGABA). In some embodiments, the composition comprises ursodeoxycholate and one or more of glutamate, malate, 5-dodecenoate, N-acetyl-L-glutamate, citrate, glycodeoxycholate, and carboxyethyl aminobutyric acid (CEGABA). In some embodiments, the composition comprises 5-dodecenoate and one or more of glutamate, malate, ursodeoxycholate, N-acetyl-L-glutamate, citrate, glycodeoxycholate, and carboxyethyl aminobutyric acid (CEGABA). In some embodiments, the composition comprises N-acetyl-L-glutamate and one or more of glutamate, malate, ursodeoxycholate, 5-dodecenoate, citrate, glycodeoxycholate, and carboxyethyl aminobutyric acid (CEGABA). In some embodiments, the composition comprises citrate and one or more of glutamate, malate, ursodeoxycholate, 5-dodecenoate, N-acetyl-L-glutamate, glycodeoxycholate, and carboxyethyl aminobutyric acid (CEGABA). In some embodiments, the composition comprises glycodeoxycholate and one or more of glutamate, malate, ursodeoxycholate, 5-dodecenoate, N-acetyl-L-glutamate, citrate, and carboxyethyl aminobutyric acid (CEGABA). In some embodiments, the composition comprises CEGABA and one or more of glutamate, malate, ursodeoxycholate, 5-dodecenoate, N-acetyl-L-glutamate, citrate, and glycodeoxycholate.

In some embodiments, the composition comprises a therapeutically effective amount of glutamate and a therapeutically effective amount of one or more of malate, ursodeoxycholate, 5-dodecenoate, N-acetyl-L-glutamate, citrate, glycodeoxycholate, and carboxyethyl aminobutyric acid (CEGABA). In some embodiments, the composition comprises a therapeutically effective amount of malate and a therapeutically effective amount of one or more of glutamate, ursodeoxycholate, 5-dodecenoate, N-acetyl-L-glutamate, citrate, glycodeoxycholate, and carboxyethyl aminobutyric acid (CEGABA). In some embodiments, the composition comprises a therapeutically effective amount of ursodeoxycholate and a therapeutically effective amount of one or more of glutamate, malate, 5-dodecenoate, N-acetyl-L-glutamate, citrate, glycodeoxycholate, and carboxyethyl aminobutyric acid (CEGABA). In some embodiments, the composition comprises a therapeutically effective amount of 5-dodecenoate and a therapeutically effective amount of one or more of glutamate, malate, ursodeoxycholate, N-acetyl-L-glutamate, citrate, glycodeoxycholate, and carboxyethyl aminobutyric acid (CEGABA). In some embodiments, the composition comprises a therapeutically effective amount of N-acetyl-L-glutamate and a therapeutically effective amount of one or more of glutamate, malate, ursodeoxycholate, 5-dodecenoate, citrate, glycodeoxycholate, and carboxyethyl aminobutyric acid (CEGABA). In some embodiments, the composition comprises a therapeutically effective amount of citrate and a therapeutically effective amount of one or more of glutamate, malate, ursodeoxycholate, 5-dodecenoate, N-acetyl-L-glutamate, glycodeoxycholate, and carboxyethyl aminobutyric acid (CEGABA). In some embodiments, the composition comprises a therapeutically effective amount of glycodeoxycholate and a therapeutically effective amount of one or more of glutamate, malate, ursodeoxycholate, 5-dodecenoate, N-acetyl-L-glutamate, citrate, and carboxyethyl aminobutyric acid (CEGABA). In some embodiments, the composition comprises a therapeutically effective amount of CEGABA and a therapeutically effective amount of one or more of glutamate, malate, ursodeoxycholate, 5-dodecenoate, N-acetyl-L-glutamate, citrate, and glycodeoxycholate.

The methods provided herein can include administering to the subject a composition that includes a bacterial species. In some embodiments, the composition comprises one or more bacterial species selected from the group consisting of: Bifidobacterium bifidum, Eggerthella lenta, Eisenbergiella massilien, Prevotella copri, Romboutsia timonensis, Blautia wexlerae, Ruminiclostridium siraeum, Bacteroides intestinalis, Faecalicatena lactaris, Dialister invisus, Ruminococcus callidus, ASV₁₅₉₇ (Faecalicatena lactaris), and ASV₈₇₆ (Dialister invisus). In some embodiments, the bacterial species is depleted in the subject as compared to a control (e.g., identified as a dysbiosis as described herein).

In some embodiments, Faecalicatena lactaris included in a composition provided herein has an amplicon sequencing variant (ASV) sequence from the 16S rRNA gene that is at least 90% (e.g. at least 91%, 92%, 93%, 94%, 95%. 96%, 97%, 98%, 99%) identical to SEQ ID NO:1 (GCAAGCGTTGTCCGGAATTACTGGGTGTAAAGGGAGC GCAGGCGGATTTGCAAGTTGGAAGTGAAACCCATGGGCTCAACCCATGGACT GCTTTCAAAACTGCAGATCTTGAGTGGTGTAGAGGTAGGCGGAATTCCCGGTG TAGCGGTGGAATGCGTAGATATCGGGAGGAACACCAGTGGCGAAGGCGGCCT ACTGGGCACTAACTGACGCTGAGGCTCGAAAGCATGGGT) (ASV₁₅₉₇). In some embodiments, Dialister invisus included in a composition provided herein has an ASV sequence from the 16S rRNA gene that is at least 90% (e.g. at least 91%, 92%, 93%, 94%, 95%. 96%, 97%, 98%, 99%) identical to SEQ ID NO:2 (GCAAGCGTTA TCCGGATTTACTGGGTGTAAAGGGAGCGTAGACGGACGGGCAAGTCTGATGT GAAAGGCAGGGGCTCAACCCCTGGACTGCATTGGAAACTGTTCATCTTGAGT GCCGGAGAGGTAAGCGGAATTCCTAGTGTAGCGGTGAAATGCGTAGATATTAG GAGGAACACCAGTGGCGAAGGCGGCTTACTGGACGGTAACTGACGTTGAGGC TCGAAAGCGTGGGG) (ASV₈₇₆).

In some embodiments, the composition comprises one or more bacterial species of a bacterial family selected from the group consisting of: Streptococcaceae, Lachnospiraceae, Ruminococcaceae, Bacteroidaceae, Butyricicoccaceae, and Pasteurellaceae. In some embodiments, the composition one or more bacterial species of a bacterial genera selected from the group consisting of: Streptococcus, Blautia, Haemophilus, Faecalibacterium, Bacteroides, Roseburia, Fusicatenibacter, Lachnospira, and Agathobaculum. In some embodiments, the composition contains Blautia wexlerae, Bacteroides vulgatus, Bacteroides ovatus, Roseburia inulinivorans, Roseburia intestinalis, Fusicatenibacter saccharivorans, and Agathobaculum butyriciproducens.

In some embodiments, the composition comprises Faecalibacterium spp. with a 16S rRNA gene that is at least 90% (e.g. at least 91%, 92%, 93%, 94%, 95%. 96%, 97%, 98%, 99%) identical to SEQ ID NO: 3 (ACAAGCGTTGTCCGGAATTA CTGGGTGTAAAGGGAGCGCAGGCGGGAGAACAAGTTGGAAGTGAAATCCAT GGGCTCAACCCATGAACTGCTTTCAAAACTGTTTTTCTTGAGTAGTGCAGAGG TAGGCGGAATTCCCGGTGTAGCGGTGGAATGCGTAGATATCGGGAGGAACAC CAGTGGCGAAGGCGGCCTACTGGGCACCAACTGACGCTGAGGCTCGAAAGT GTGGGT).

In some embodiments, the composition comprises Bacteroides vulgatus with a 16S rRNA gene that is at least 90% (e.g. at least 91%, 92%, 93%, 94%, 95%. 96%, 97%, 98%, 99%) identical to SEQ ID NO: 4 (CCGAGCGTTATCCGGATTTATTGGGT TTAAAGGGAGCGTAGATGGATGTTTAAGTCAGTTGTGAAAGTTTGCGGCTCA ACCGTAAAATTGCAGTTGATACTGGATATCTTGAGTGCAGTTGAGGCAGGCG GAATTCGTGGTGTAGCGGTGAAATGCTTAGATATCACGAAGAACTCCGATTG CGAAGGCAGCCTGCTAAGCTGCAACTGACATTGAGGCTCGAAAGTGTGGGT).

In some embodiments, the composition comprises Bacteroides ovatus with a 16S rRNA gene that is at least 90% (e.g. at least 91%, 92%, 93%, 94%, 95%. 96%, 97%, 98%, 99%) identical to SEQ ID NO: 5 (CCGAGCGTTATCCGGATTTATTGGGTTT AAAGGGAGCGTAGATGGATGTTTAAGTCAGTTGTGAAAGTTTGCGGCTCAAC CGTAAAATTGCAGTTGATACTGGATATCTTGAGTGCAGTTGAGGCAGGCGGA ATTCGTGGTGTAGCGGTGAAATGCTTAGATATCACGAAGAACTCCGATTGCG AAGGCAGCCTGCTAAGCTGCAACTGACATTGAGGCTCGAAAGTGTGGGT).

In some embodiments, the composition comprises Roseburia inulinivorans with a 16S rRNA gene that is at least 90% (e.g. at least 91%, 92%, 93%, 94%, 95%. 96%, 97%, 98%, 99%) identical to SEQ ID NO: 6 (GCAAGCGTTATCCGGATTTA CTGGGTGTAAAGGGAGCGCAGGCGGAAGGCTAAGTCTGATGTGAAAGCCCG GGGCTCAACCCCGGTACTGCATTGGAAACTGGTCATCTAGAGTGTCGGAGGG GTAAGTGGAATTCCTAGTGTAGCGGTGAAATGCGTAGATATTAGGAGGAACA CCAGTGGCGAAGGCGGCTTACTGGACGATAACTGACGCTGAGGCTCGAAAGC GTGGGG). In some embodiments, the composition comprises Roseburia intestinalis with a 16S rRNA gene that is at least 90% (e.g. at least 91%, 92%, 93%, 94%, 95%. 96%, 97%, 98%, 99%) identical to SEQ ID NO: 7 (GCAAGCGTTATCCGGATTTACT GGGTGTAAAGGGAGCGCAGGCGGTACGGCAAGTCTGATGTGAAAGCCCGGG GCTCAACCCCGGTACTGCATTGGAAACTGTCGGACTAGAGTGTCGGAGGGGT AAGTGGAATTCCTAGTGTAGCGGTGAAATGCGTAGATATTAGGAGGAACACC AGTGGCGAAGGCGGCTTACTGGACGATTACTGACGCTGAGGCTCGAAAGCGT GGGG).

In some embodiments, the composition comprises Faecalicatena torques with a 16S rRNA gene that is at least 90% (e.g. at least 91%, 92%, 93%, 94%, 95%. 96%, 97%, 98%, 99%) identical to SEQ ID NO: 8 (GCAAGCGTTATCCGGATTTACT GGGTGTAAAGGGAGCGTAGACGGATGGGCAAGTCTGATGTGAAAACCCGGG GCTCAACCCCGGGACTGCATTGGAAACTGTTCATCTAGAGTGCTGGAGAGGT AAGTGGAATTCCTAGTGTAGCGGTGAAATGCGTAGATATTAGGAGGAACACC AGTGGCGAAGGCGGCTTACTGGACAGTAACTGACGTTGAGGCTCGAAAGCGT GGGG).

In some embodiments, the composition comprises Fusicatenibacter saccharivorans with a 16S rRNA gene that is at least 90% (e.g. at least 91%, 92%, 93%, 94%, 95%. 96%, 97%, 98%, 99%) identical to SEQ ID NO: 9 (GCAAGCG TTATCCGGATTTACTGGGTGTAAAGGGAGCGTAGACGGCAAGGCAAGTCTGA TGTGAAAACCCAGGGCTTAACCCTGGGACTGCATTGGAAACTGTCTGGCTCG AGTGCCGGAGAGGTAAGCGGAATTCCTAGTGTAGCGGTGAAATGCGTAGATA TTAGGAAGAACACCAGTGGCGAAGGCGGCTTACTGGACGGTAACTGACGTTG AGGCTCGAAAGCGTGGGG).

In some embodiments, the composition comprises Lachnospira spp. with a 16S rRNA gene that is at least 90% (e.g. at least 91%, 92%, 93%, 94%, 95%. 96%, 97%, 98%, 99%) identical to SEQ ID NO: 10 (GCAAGCGTTATCCGGATTTACTGGGTG TAAAGGGAGTGTAGGTGGCCATGCAAGTCAGAAGTGAAAATCCGGGGCTCA ACCCCGGAACTGCTTTTGAAACTGTAAGGCTAGAGTGCAGGAGGGGTGAGTG GAATTCCTAGTGTAGCGGTGAAATGCGTAGATATTAGGAGGAACACCAGTGG CGAAGGCGGCTCACTGGACTGTAACTGACACTGAGGCTCGAAAGCGTGGGG).

In some embodiments, the composition comprises a species of the Lachnospiraceae family with a 16S rRNA gene that is at least 90% (e.g. at least 91%, 92%, 93%, 94%, 95%. 96%, 97%, 98%, 99%) identical to SEQ ID NO: 11 (GCAAGCGTTATCCGGATTTACTGGGTGTAAAGGGAGTGTAGGTGGTATCACA AGTCAGAAGTGAAAGCCCGGGGCTCAACCCCGGGACTGCTTTTGAAACTGTG GAACTGGAGTGCAGGAGAGGTAAGTGGAATTCCTAGTGTAGCGGTGAAATGC GTAGATATTAGGAGGAACACCAGTGGCGAAGGCGGCTTACTGGACTGTAACT GACACTGAGGCTCGAAAGCGTGGGG).

In some embodiments, the composition comprises Agathobaculum butyriciproducens with a 16S rRNA gene that is at least 90% (e.g. at least 91%, 92%, 93%, 94%, 95%. 96%, 97%, 98%, 99%) identical to SEQ ID NO: 12 (GCAAGCG TTATCCGGATTTACTGGGTGTAAAGGGCGCGCAGGCGGGCCGGCAAGTTGGA AGTGAAATCTATGGGCTTAACCCATAAACTGCTTTCAAAACTGCTGGTCTTGA GTGATGGAGAGGCAGGCGGAATTCCGTGTGTAGCGGTGAAATGCGTAGATAT ACGGAGGAACACCAGTGGCGAAGGCGGCCTGCTGGACATTAACTGACGCTG AGGCGCGAAAGCGTGGGG) or SEQ ID NO: 13 (GCAAGCGTTATCCGGATTTA CTGGGTGTAAAGGGCGCGCAGGCGGGCCGGTAAGTTGGAAGTGAAATCTATG GGCTTAACCCATAAACTGCTTTCAAAACTGCTGGTCTTGAGTGATGGAGAGG CAGGCGGAATTCCGTGTGTAGCGGTGAAATGCGTAGATATACGGAGGAACAC CAGTGGCGAAGGCGGCCTGCTGGACATTAACTGACGCTGAGGCGCGAAAGC GTGGGG).

In some embodiments, the method can include detecting, in a sample from the subject, a dysbiosis associated with autism spectrum disorder, e.g., before administering to the subject a metabolite described herein or a bacterial species described herein. In some embodiments, the sample is a biological sample. In some embodiments, the sample is a fecal (stool) sample, a sputum sample, a saliva sample, a mucous sample, a nasal sample, a nasopharyngeal sample, an oral sample, or a respiratory fluid sample. In some embodiments, the sample is a fecal sample or a stool sample.

In some embodiments, detecting the dysbiosis associated with autism spectrum disorder can include determining a bacterial gene and its expression in the sample from the subject (e.g., fecal sample). For example, the bacterial gene and its expression can be determined in the sample from the subject e.g., before administering to the subject a metabolite described herein or a bacterial species described herein and/or after administering to the subject a metabolite described herein or a bacterial species described herein. Determining the bacterial gene and its expression can include performing, for example, RNAseq and/or RT-qPCR. In some embodiments, detecting the dysbiosis associated with autism spectrum disorder comprises determining bacterial composition in the sample from the subject (e.g., fecal sample). For example, the bacterial composition can be determined in a sample from the subject, e.g., before administering to the subject a metabolite described herein or a bacterial species described herein and/or after administering to the subject a metabolite described herein or a bacterial species described herein. Determining the bacterial composition can include, for example, sequencing one or more nucleic acids from the bacteria or the sample (e.g. fecal or stool sample). In some embodiments, bacteria can be identified by their 16S rRNA gene sequence.

In some embodiments, detecting the dysbiosis associated with autism spectrum disorder comprises determining that a bacterial species from the Pasteurellaceae family, Ruminococcaceae family, Bacteroidaceae family, Butyricicoccaceae family, Streptococcus genus, Blautia genus, Haemophilus genus, Faecalibacterium genus, Bacteroides genus, Roseburia genus, Fusicatenibacter genus, Lachnospira genus, Agathobaculum genus or a combination thereof that is depleted in the sample from the subject (e.g., reduced in the fecal sample or reduced in the gastrointestinal tract of the subject). In some embodiments, the bacterial species that is depleted in the sample from the subject is selected from the group consisting of: Blautia wexlerae, Bacteroides vulgatus, Bacteroides ovatus, Roseburia inulinivorans, Roseburia intestinalis, Fusicatenibacter saccharivorans, and Agathobaculum butyriciproducens. In some embodiments, the methods can include administering the depleted bacterial species to the subject.

In some embodiments, detecting the dysbiosis associated with autism spectrum disorder comprises determining that a bacterial species from the Bacteroidaceae family, Lachnospiraceae family, Oscillospiraceae family, Anaerovoraceae family, Erysipelotrichaceae family, Christensenellaceae family, Bacteroides genus, Blautia genus, Holdemania genus, Borkfalki genus, Anaerotignum genus, Faecalicatena genus, or a combination thereof is enriched in the sample from subject (e.g., increased in the fecal sample or increased in the gastrointestinal tract of the subject). In some embodiments, the bacterial species that is enriched in the sample from the subject is selected from the group consisting of: Bacteroides thetaiotaomicron, Borfalki ceftriaxensis, and Faecalicatena torques. In some embodiments, the methods can include administering a treatment to deplete a species that was enriched in the subject, e.g., using antibiotics or phage that are specific to the strain, species, or genera.

In some embodiments, the methods provided herein can include administering a composition described herein (e.g., a composition comprising a metabolite described herein or bacterial species described herein) to the subject at least once per day. For example, the composition can be administered two, three, four, or more times per day. In some embodiments, the method comprises administering a composition described herein (e.g., a composition comprising a metabolite described herein or bacterial species described herein) to the subject daily, every other day, every three days, or once a week.

In some embodiments, an effective amount of a metabolite described herein or bacterial species described herein is administered in one dose, e.g., once per day. In some embodiments, an effective amount of the metabolite described herein or bacterial species described herein is administered in more than one dose, e.g., more than once per day.

In some embodiments, methods provided herein can include administering a composition described herein (e.g., a composition comprising a metabolite described herein or bacterial species described herein) in combination with one or more other treatments of ASD. Non-limiting examples of other treatments of ASD include: antipsychotic drugs, antidepressants, behavioral therapy, psychological therapy, educational therapy, occupational therapy, and speech therapy. Compositions described herein (e.g., a composition comprising a metabolite described herein or bacterial species described herein) and any other treatments can be administered together (e.g., in the same formulation), or the composition comprising the bacterial species can be administered concurrently with, prior to, or subsequent to, the one or more other treatments.

In some embodiments, a prebiotic and/or probiotic can be administered in combination with a composition described herein (e.g., a composition comprising a metabolite described herein or bacterial species described herein). Non-limiting examples of a probiotic include one of more of Bifidobacteria (e.g., B. animalis, B. breve, B. lactis, B. longum, B. longum, or B. infantis), Lactobacillus (e.g., L. acidophilus, L. reuteri, L. bulgaricus, L. lactis, L. casei, L. rhamnosus, L. plantarum, L. paracasei, or L. delbrueckii/bulgaricus), Saccharomyces boulardii, E. coli Nissle 1917, and Streptococcus thermophiles. Non-limiting examples of a prebiotic include a fructooligosaccharide (e.g., oligofructose, inulin, or an inulin-type fructan), a galactooligosaccharide, an amino acid, or an alcohol. See, for example, Ramirez-Farias et al. (2008. Br. J Nutr. 4:1-10) and Pool-Zobel and Sauer (2007. J Nutr. 137:2580-2584).

In some embodiments, methods provided herein can include monitoring the subject after treatment with a composition described herein (e.g., a composition comprising a metabolite described herein or bacterial species described herein) to determine if one or more symptoms have been alleviated, if the severity of one or more symptoms has been reduced, or if progression of the disease has been delayed or inhibited in the subject. Non-limiting examples of autism spectrum disorder symptoms include: making little or inconsistent eye contact; tending not to look at or listen to people; rarely sharing enjoyment of objects or activities by pointing or showing things to others; failing to, or being slow to, respond to someone calling their name or to other verbal attempts to gain attention; having difficulties with the back and forth of conversation; often talking at length about a favorite subject without noticing that others are not interested or without giving others a chance to respond; having facial expressions, movements, and gestures that do not match what is being said; having an unusual tone of voice that may sound sing-song or flat and robot-like; having trouble understanding another person's point of view or being unable to predict or understand other people's actions; repeating certain behaviors or having unusual behaviors (e.g., repeating words or phrases (echolalia)); having a lasting intense interest in certain topics, such as numbers, details, or facts; having overly focused interests, such as with moving objects or parts of objects; getting upset by slight changes in a routine; and being more or less sensitive than other people to sensory input, such as light, noise, clothing, or temperature.

Is some embodiments, methods provided herein can include monitoring the subject after treatment with a composition described herein (e.g. a composition comprising a metabolite described herein or a bacterial species described herein) to determine if one or more comorbidities have been alleviated or if the severity of one or more comorbidities has been reduced. Non-limiting examples of autism spectrum disorder comorbidities include: intellectual disability, epilepsy, anxiety, sleep, and gastrointestinal disorders. See Cheroni et al. Mol Autism. 2020; 11: 69. There are numerous scores and clinical markers that can be utilized to assess the efficacy of administering a composition described herein (e.g., a composition comprising a metabolite described herein or bacterial species described herein) in treating ASD. In some embodiments, the subject has severe autism. In some embodiments, the autism severity is measured using the Mobile Autism Risk Assessment (MARA). See, e.g., Duda et al. J Autism Dev Disord. 2016; 46: 1953-1961.

In some embodiments, methods provided herein can include administering another treatment for autism spectrum disorder to the subject. Non-limiting examples of treatments can include medication, such as antipsychotics (e.g., risperidone or aripiprazole) or stimulants (e.g. methylphenidate, atomoxetine, or clonidine), or therapy, such as behavioral therapy, family counseling, speech and/or language therapy, or educational therapy.

In some embodiments, the compositions described herein (e.g., compositions comprising a metabolite described herein or bacterial species described herein) can include one or more excipients and can be formulated for any of a number of delivery systems suitable for administration to a subject. Non-limiting examples of an excipient include a buffering agent, a diluent, a preservative, a stabilizer, a binding agent, a filler, a lubricant, a dispersion enhancer, a disintegrant, a lubricant, wetting agent, a glidant, a flavoring agent, a sweetener, and a coloring agent. For example, in some embodiments, tablets or capsules can be prepared by conventional means with excipients such as binding agents, fillers, lubricants, disintegrants, or wetting agents. Any of the compositions described herein can be administered to a subject to treat ASD as described herein.

In some embodiments, a composition as described herein (e.g., a composition comprising a metabolite described herein or bacterial species described herein) can be formulated for oral delivery. In some embodiments, the composition can be formulated as a tablet, a chewable tablet, a capsule, a stick pack, a powder, effervescent powder, or a liquid. In some embodiments, the composition can be formulated as a tablet. In some embodiments, the tablet is coated, for example, the tablet is coated with an enteric coating. In some embodiments, the tablet is coated with a coating for timed release. In some embodiments, the table is coated with a coating for immediate release. In some embodiments, the tablet is not coated.

In some embodiments, a composition can include coated beads that contain a metabolite described herein or a bacterial species described herein. In some embodiments, a powder comprising the metabolite or bacterial species can be suspended or dissolved in a drinkable liquid such as water for administration. In some embodiments, the composition is a solid composition.

In some embodiments, a composition described herein (e.g., compositions comprising a metabolite described herein or bacterial species described herein) can be formulated for various immediate and controlled release profiles of the metabolite or bacterial species. For example, a controlled release formulation can include a controlled release coating disposed over the metabolite or bacterial species. In some embodiments, the controlled release coating is an enteric coating, a semi-enteric coating, a delayed release coating, or a pulsed release coating. In some embodiments, a coating can be suitable if it provides an appropriate lag in active release (i.e., release of the metabolite or bacterial species). For example, in some embodiments, the composition can be formulated as a tablet that includes a coating (e.g., an enteric coating).

The invention will be further described in the following examples, which do not limit the scope of the invention described in the claims.

### EXAMPLES

### Example 1. Identification of Metabolites Associated with Autism Spectrum Disorder

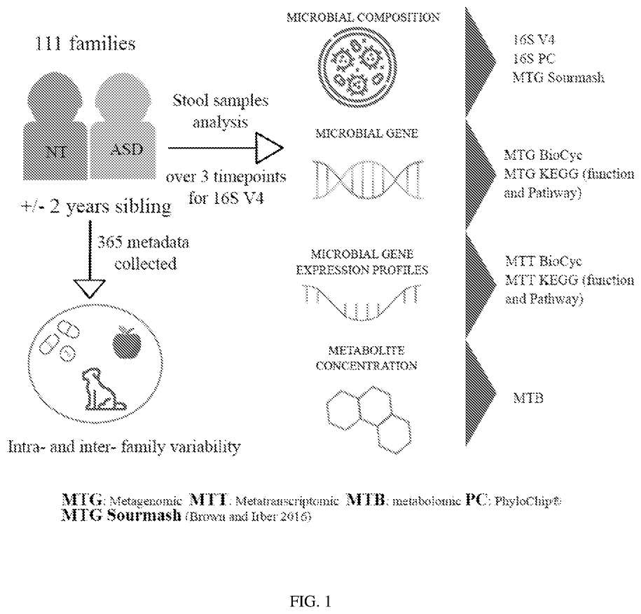

The M3 consortium (Metabolite, Microbiome and the Mind) recruited a large cohort of 111 families with one ASD child and one neurotypical (NT) sibling in the same age range to minimize the impact of genetics, diet and environment. Severity of autism in ASD subjects was captured using the Mobile Autism Risk Assessment (MARA). In addition, 365 metadata features assessing inter- and intra-family variabilities were collected to permit further investigation of environmental factors' influences on the ASD microbiome. Gut microbiome from stool samples was characterized at the DNA, RNA and metabolite levels using multi-omics technologies, including, 16S V4 rRNA region next generation sequencing (16S NGS), 16S V1-V9 rRNA PhyloChip® DNA micro-array (16S PC), whole-metagenome shotgun sequencing (MTG), metatranscriptomics (MTT) and metabolomics (MTB). See FIG. 1.

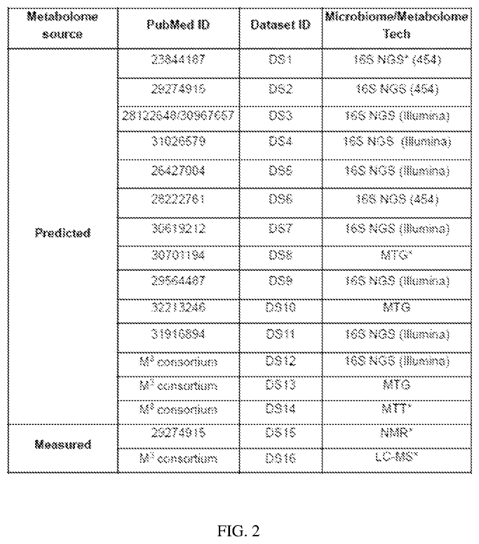

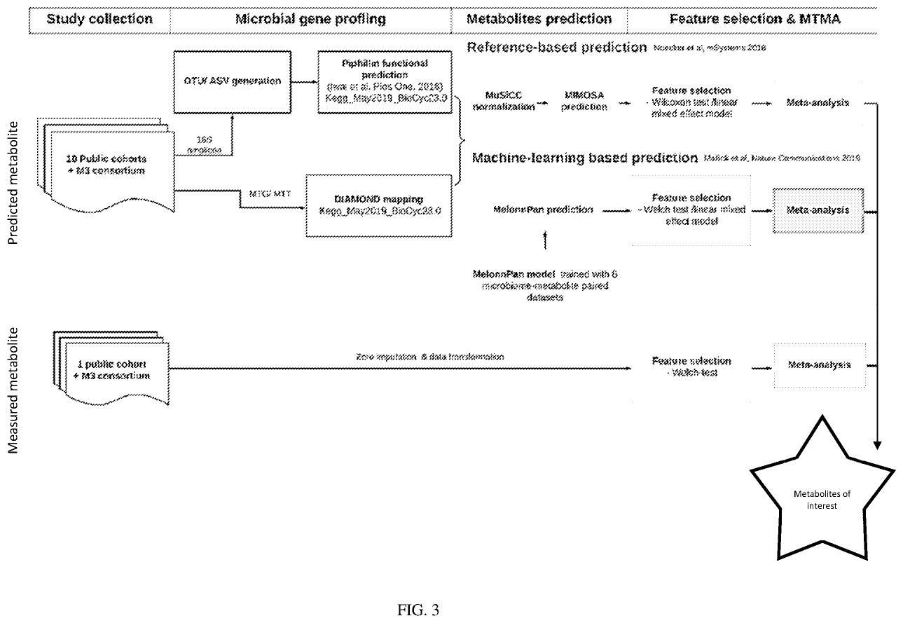

Multi-Technology Meta-Analysis (MTMA) was used to identify ASD-associated metabolites for drug development by combining both in silico prediction and empirical metabolite measurement data followed by meta-analysis using 11 ASD cohorts including the M3 consortium (FIG. 2). In silico metabolome prediction was performed using the microbiome sequencing data of the fecal samples from these subjects. Both a reference-based, gene-to-metabolite prediction strategy, and a machine-learning based strategy using a newly trained model were employed for prediction. The resulting predictions for the metabolome, as well as the observed measured metabolome data, were analyzed to identify differential microbial metabolites associated with ASD followed by meta-analysis. See FIG. 3.

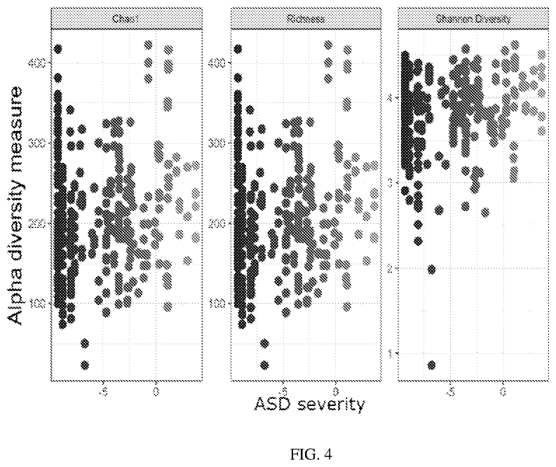

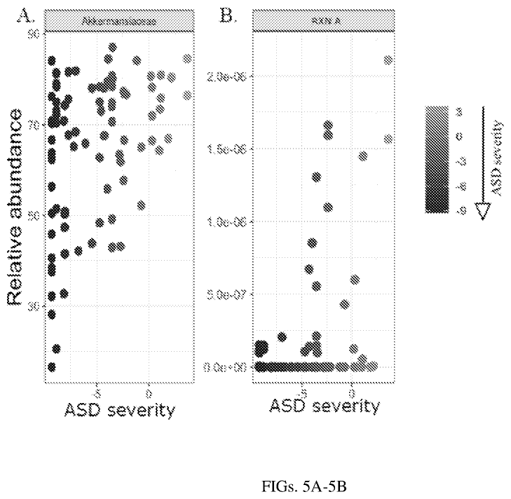

ASD severity correlated with microbial composition and functionality. As shown in FIG. 4 there was significantly reduced alpha diversity for the most severe cases of ASD (Spearman test with pvalue<0.05 in 16S V4 data set). As shown in FIG. 5, there was significantly reduced relative abundance (Spearman test with p_{adj}<0.05 and |Rho|>0.3) of Akkermansiaceae family (16S PC) (FIG. 5A) and RXN A (reaction A; MTG BioCyc; 3.2.1.132-RXN (EC 3.2.1.132=chitosanase) Chitosanase catalyzes the endohydrolysis of beta-(1->4)-linkages between D-glucosamine residues in a partly acetylated chitosan) (FIG. 5B) for the most severe cases of ASD. The metabolite associated to RXN A (chitosan) has been reported as an important molecule against dysbiosis. See, e.g., Wang, Jia, Cuili Zhang, Chunmei Guo, and Xinli Li. 2019. “Chitosan Ameliorates DSS-Induced Ulcerative Colitis Mice by Enhancing Intestinal Barrier Function and Improving Microflora.” International Journal of Molecular Sciences 20 (22). doi.org/10.3390/ijms20225751; Qian, Minyi, Qianqian Lyu, Yujie Liu, Haiyang Hu, Shilei Wang, Chuyue Pan, Xubin Duan, et al. 2019. “Chitosan Oligosaccharide Ameliorates Nonalcoholic Fatty Liver Disease (NAFLD) in Diet-Induced Obese Mice.” Marine Drugs 17 (7). doi.org/10.3390/md17070391; Zheng, Junping, Xubing Yuan, Gong Cheng, Siming Jiao, Cui Feng, Xiaoming Zhao, Heng Yin, Yuguang Du, and Hongtao Liu. 2018. “Chitosan Oligosaccharides Improve the Disturbance in Glucose Metabolism and Reverse the Dysbiosis of Gut Microbiota in Diabetic Mice.” Carbohydrate Polymers 190 (June): 77-86. doi.org/10.1016/j.carbpol.2018.02.058; and Gao, Jing, Md A. K. Azad, Hui Han, and Dan Wan and TieJun Li. 2020. “Impact of Prebiotics on Enteric Diseases and Oxidative Stress.” Current Pharmaceutical Design. May 31, 2020. eurekaselect.com/179241/article. Reduced alpha diversity might cause perturbation in the gut microbiome of the subject and accentuate the symptoms. Reduced abundance of Akkermansiaceae family is associated with ASD severity. This could indicate a thinner GI mucus barrier in children with severe ASD compared to the others. The result might reflect an indirect evidence of impaired gut permeability in children with severe ASD (Wang et al. 2011. Applied and Environmental Microbiology. 77:18, 6718-6721).

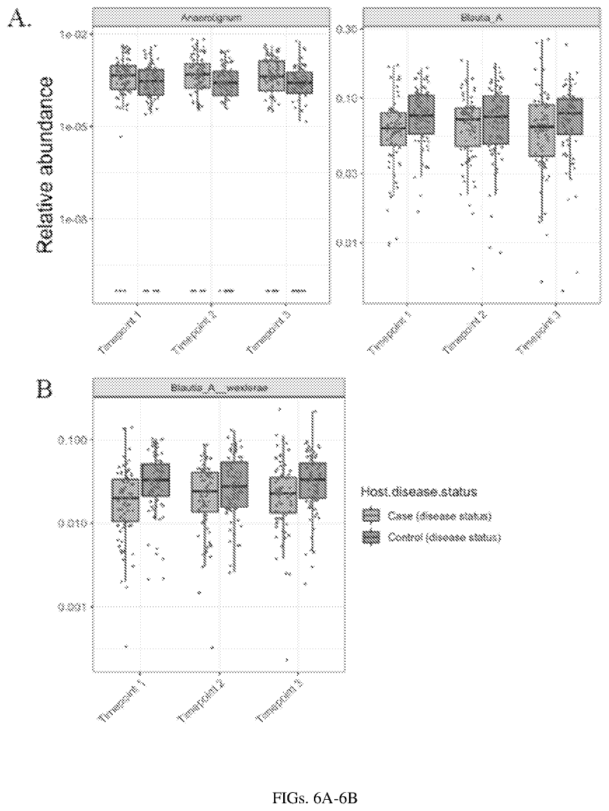

Significant differences in bacterial and metabolite composition between ASD and NT might underlie gastro-intestinal and neurodevelopmental symptoms in ASD. FIG. 6 shows the relative abundance of significantly different taxa between ASD and NT groups across the 3 time-points with FIG. 6A at the genus level and FIG. 6B at the species level (Kruskall Wallis test paired by family ID with p_{adj}<0.05 in the 16S V4 data set).

Anaerotignum genus consistently showed higher abundance in ASD across three timepoints and Blautia genus showed an opposite trend across the 3 time points. Blautia has been reported in several ASD studies as depleted in ASD subjects compared to NT. The decrease in Blautia genus detected in ASD children may be associated with constipation, which seems to provide evidence of the presence of gut dysbiosis (Inoue et al., 2019). Blautia wexlerae species was observed to be consistently lower abundance in ASD across three time points. This species shows anti-inflammatory properties (Benítez-Páez et al., 2020. mSystems. 5:2, e00857-19).

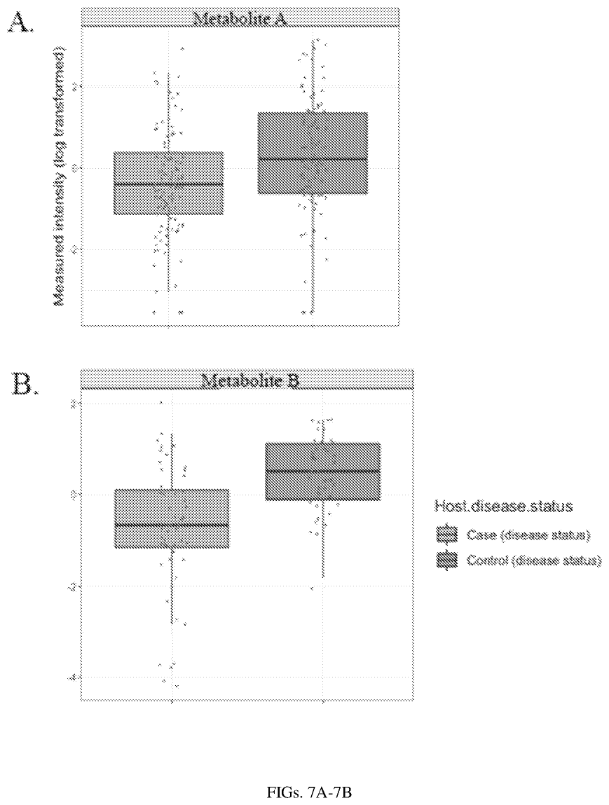

FIG. 7 shows the measured intensity between the ASD and NT groups of Metabolite A (5-dodecenoate) (FIG. 7A) and Metabolite B (CEGABA) (FIG. 7B). Welch test was performed on log 2 transformed data with zero was imputed as the minimum value per metabolite.

Metabolite A is a mono-unsaturated fatty acid (MUFA). Other studies indicate a link between autism and MUFA, Bell et al. found that total MUFA were significantly reduced in regressive autism patients compared with controls (Bell et al. 2010).

Metabolite B is the intermediate of an alternative metabolic pathway in the biosynthesis of a neuromodulator. Abnormalities in the signaling of this neuromodulator have been hypothesized to underlie ASD symptoms. This neurotransmitter was found depleted in other ASD studies.

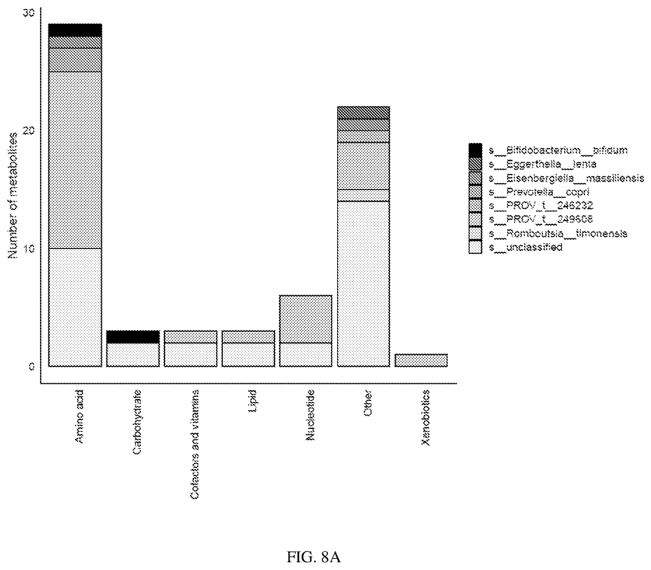

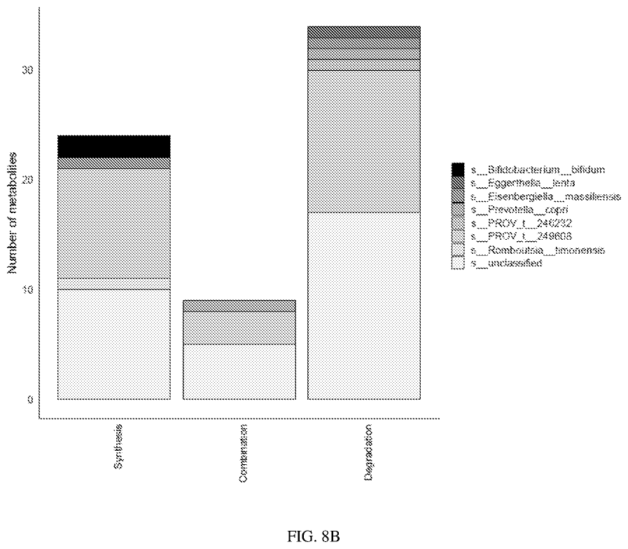

FIG. 8 shows the results of linking variation in metabolomic data with community composition by utilizing taxonomic, genomic, and metabolic information (Noecker et al. 2016. mSystems. 1:1, e00013-15). FIG. 8A are the putative bacterial contributors to variation in amino acids and other metabolites, and FIG. 8B is a summary of bacterial contribution type of the putative bacterial contributors. The gene abundance from the microbial composition (16S V4) was predicted utilizing the software Piphillin (Iwai et al. 2016. Plos One. 11:11, e0166104; Narayan et al. 2020. BMC Genomics. 21:56, doi.org/10.1186/s12864-019-6427-1) using a 99% ID cutoff and normalized utilizing the MUSiCC algorithm resulting in an estimate of the average copy number of each gene across microbiome genomes (Manor and Borenstein 2015. Genome biology. 16:53, doi.org/10.1186/s13059-015-0610-8).

The highest number of metabolites from Amino acid category were contributed to microbial activities. An unnamed species within the Ruminococcaceae family and within the Gemmiger genus, and the species Prevotella copri contributed to the most number of metabolites.

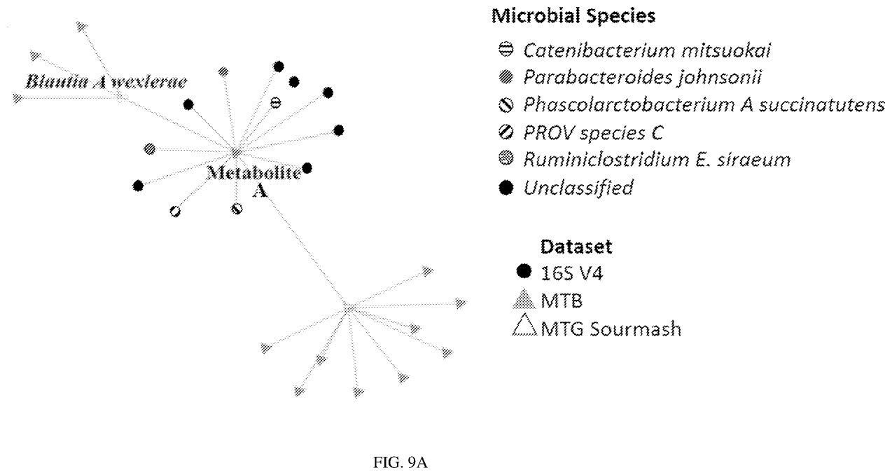

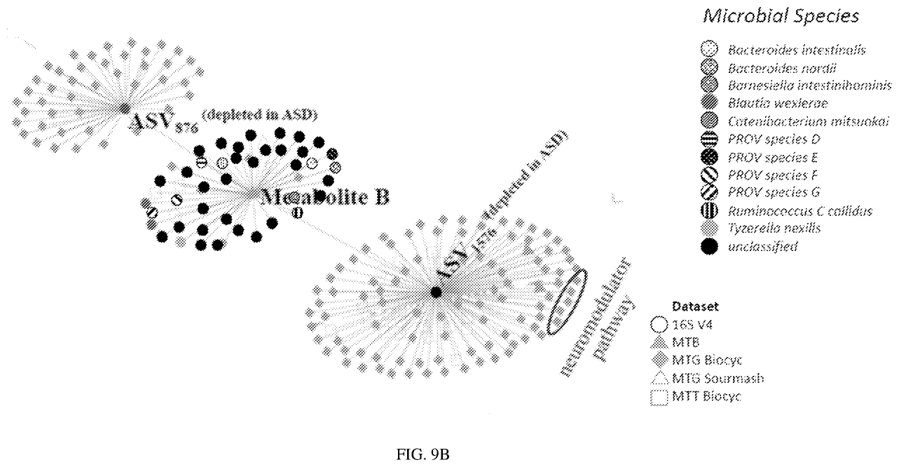

FIG. 9 shows pairwise correlation using Spearman test (Padj<0.05 and |Rho|>0.3) have been used to determine the significant correlation between the data sets and to build networks around Metabolite A (FIG. 9A) and Metabolite B (FIG. 9B). Metabolite A significantly correlated with Blautia wexlerae, which is significantly depleted in the ASD group. Metabolite B significantly correlated with ASV 1597, which further associated with microbial genes involved in neuromodulator pathway.

The following metabolites were identified:
1. Glutamate: identified from sg_project_id UNFII_FLembo_BIRD18_0289 16S sequencing data based on the MelonnPan pipeline (Mallick et al., 2019) when using BioCyc as the reference database, with P value=3.74E−06, P adjust value=5.53E−05 and effect size in Log 2FoldChange (ASD/NT)=−2.5 by Welch test followed by Benjamini-Hochberg adjustment.
2. Malate: identified from sg_project_id UNFII_FLembo_BIRD18_0289 16S sequencing data based on the MelonnPan pipeline when using BioCyc and KEGG as the reference database. When using BioCyc as the reference database, P value=2.49E−06, P adjust value=3.96E−05 and Effect size in Log 2FoldChange (ASD/NT)=−3.3; When using KEGG as the reference database, P value=1.81E−06, P adjust value=3.18E−05 and effect size in Log 2FoldChange (ASD/NT)=−2.5 by Welch test followed by Benjamini-Hochberg adjustment.
3. Ursodeoxycholate: identified from sg_project_id UNFII_FLembo_BIRD18_0289 16S sequencing data based on the MelonnPan pipeline when using BioCyc as the reference database, with P value=1.85E−06, P adjust value=3.82E−05 and effect size in Log 2FoldChange (ASD/NT)=−1.3 by Welch test followed by Benjamini-Hochberg adjustment.
4. 5-dodecenoate (12:1n7): identified from M3 Metabolome data, with P value=1.17E−05, P adjust value=0.01 and effect size in Log 2FoldChange (ASD/NT)=−0.8 by Welch test followed by Benjamini-Hochberg adjustment.
5. N-acetyl-L-glutamate: identified from sg_project_id UNFII_FLembo_BIRD18_0289 16S sequencing data based on the MelonnPan pipeline when using BioCyc as the reference database, with P value=6.65E−06, P adjust value=7.18E−05 and effect size in Log 2FoldChange (ASD/NT)=−2.5 by Welch test followed by Benjamini-Hochberg adjustment.
6. Citrate: identified from meta-analysis from MelonnPan pipeline when using BioCyc as the reference database. P value=0.08, P adjust value=0.63 and effect size in Log 2FoldChange (ASD/NT)=−0.4 by Welch test followed by Benjamini-Hochberg adjustment.
7. Glycodeoxycholate: identified from meta-analysis from MelonnPan pipeline when using BioCyc as the reference database with P value=0.04, P adjust value=0.63 and effect size in Log 2FoldChange (ASD/NT)=−0.04 by Welch test followed by Benjamini-Hochberg adjustment.
8. CEGABA: Carboxyethyl aminobutyric acid (CEGABA) has been found depleted in the most severe cases of Autism Spectrum Disorder (51 subjects with a Mobile Autism Risk Assessment score <8) compared to their neurotypical siblings in the M3 study. A Welch test (paired by family ID) was performed on log 2 transformed data with zero imputed as the minimum value per metabolite to access differentially abundant metabolites between the groups. Pvalue=2.43e−5, Padjusted value=0.0233 (Padjusted value calculated with Benjamini-Hochberg procedure) and effect size in Log 2FoldChange=−1.18

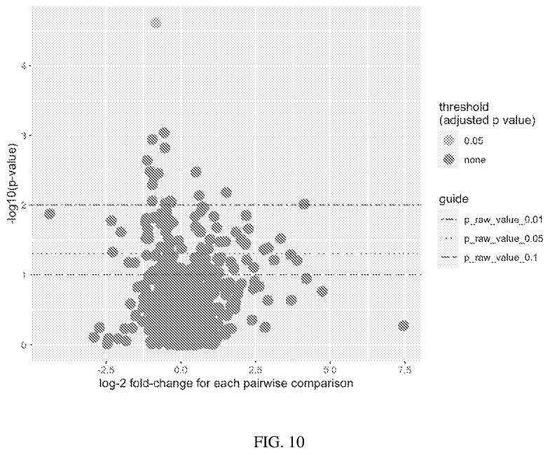

FIG. 10 shows a volcano plot of the results of the Welch test (paired by family ID) comparing the metabolites abundance in the most severe cases of ASD and their neurotypical siblings. CEGABA was the most significantly different metabolite. See FIG. 7B.

Comparing differences in ASD and NT, significant differences were found in compositions of 16S NGS at strain levels (Wald test) as well as higher taxonomical levels (Wilcoxon rank sum test) but not by 16S PC or MTG. Specific KEGG or BioCyc functional differences between ASD and NT in MTG and MTT were also identified. In addition, bacterial composition and KEGG or BioCyc functional differences associated with severity (MARA score) of the ASD subjects (Spearman's rank correlation and Wald test) were identified. Further integrative analysis revealed specific microbial taxa and KEGG or BioCyc functions that are correlated with one metabolite that was significantly less abundant in ASD compared to NT subjects.

### Example 2. Effects of Metabolites Associated with Autism Spectrum Disorder on Gene Transcription in Brain Tissue in Mice

Metabolites identified in Example 1 were use in experiments to determine if the metabolites affected gene transcription patterns in the mouse brain or mouse behavior.

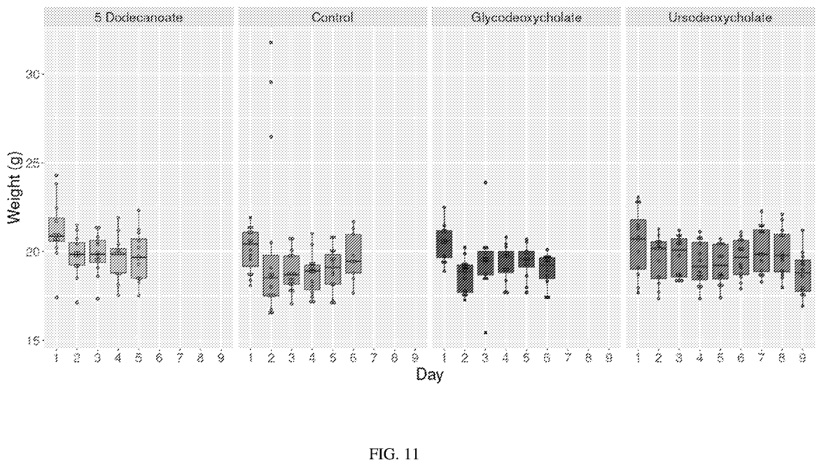

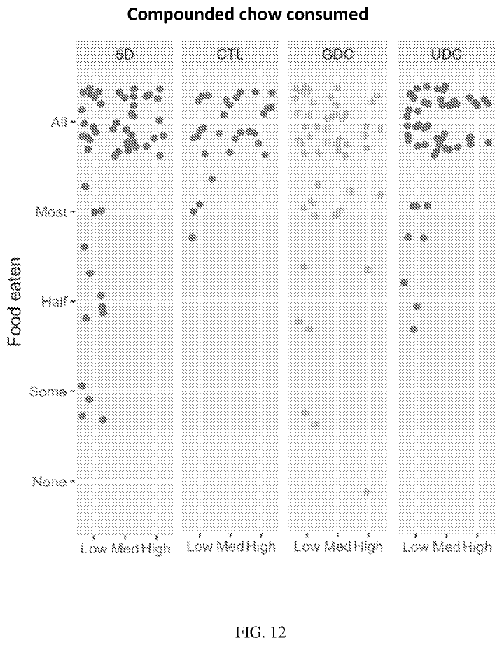

Mice were fed food mixed with 5-dodecanoate (5D), glycodeoxycholate (GDC), ursodeoxycholate (UDC), or control food (CTL) that did not have a supplemented metabolite. Mouse weighs and the amount of food (chow) consumed were recorded daily. The mouse's weight on day 1 was the initial control weight. Feeding with mixed food began on day 2. A large decrease in weight for all mice was observed between day 1 and 2 likely due to cage changes, handling, and new food placement. There was no any significant decrease in mouse weight indicating that these compounds are safe (FIG. 11). Additionally, the identity of the metabolite mixed with mouse food did not affect how much food was consumed by mice (FIG. 12).

Transcriptomics data (RNA sequencing) was collected from the prefrontal cortex of 12-week old mice after consuming a low, medium, or high dosage of supplemented metabolites for two weeks. RNA sequencing used brain tissue to evaluate possible correlation between changes in relative changes in gene transcription with changes in behavior. The number of differentially expressed gene pathways at significance level of 0.05, 0.1, and 0.15 was determined (Table 1). The expression of many genes changed in mouse brain tissue due to supplementation with various metabolites or compounds. No changes in expression were observed in brain tissue from mice fed control food lacking a supplement.

TABLE 1
Differentially Expressed Gene Pathways in Mouse Brain After
Consuming Food Supplemented with Various Metabolites.

| | | | |
|---|---|---|---|
| Metabolite | 0.05 | 0.1 | 0.15 |
| 5-Dodecenoate | 0 | 216 | 235 |
| Control | 0 | 0 | 0 |
| Glutamate | 0 | 0 | 107 |
| Glycodeoxycholate | 153 | 277 | 291 |
| N-acetyl-L- | 82 | 145 | 177 |
| glutamate | | | |
| Ursodeoxycholate | 0 | 0 | 104 |

### Example 3. Testing the Effect of Metabolites Associated with Autism Spectrum Disorder on Behavior of Mice

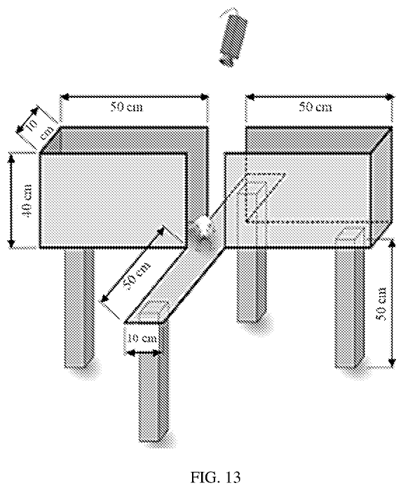

Mice were fed 80% of their total food as non-supplemented food (chow) for 4 days. For the next two weeks, mice were fed 80% of their total food as non-supplemented food and the remaining 20% was supplemented with selected metabolites(s). Mice were fed on food supplemented with various metabolites including 5-dodecacenoate (5D), glycodeoxycholate (GDC), ursodeoxycholate (UDC), or a control food lacking a supplement (CTL). Mouse behavior was tested using an elevated plus maze (FIG. 13), a three-chamber sociability test, and tracking wheel usage. During testing, mice were fed 80% of their total food as non-supplemented food and the remaining 20% was supplemented with the selected metabolites(s).

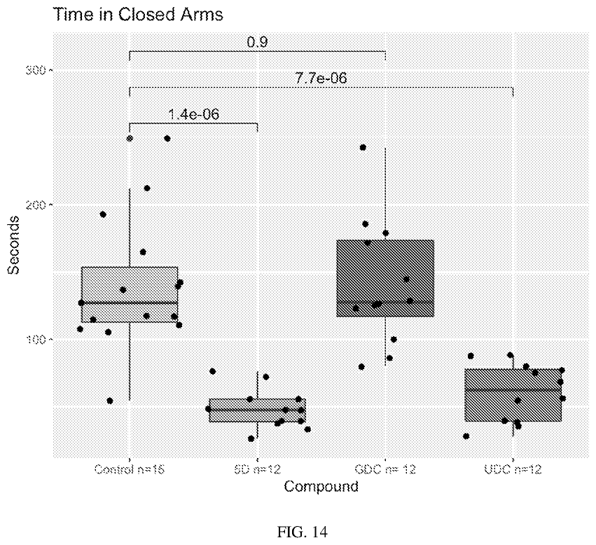

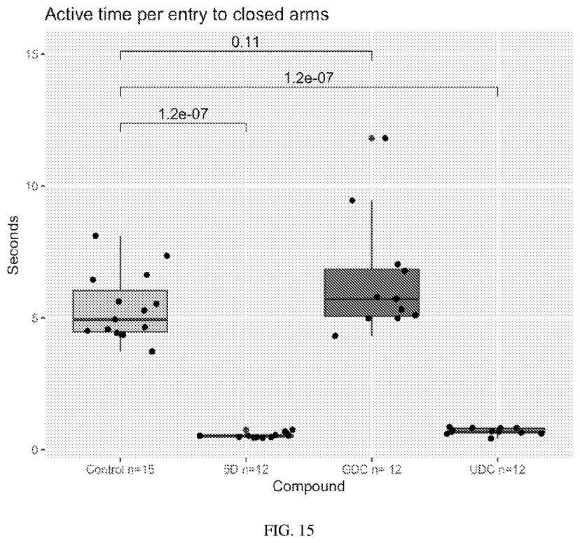

Anxiogenic behavior was tested by tracking time spent in closed arms of an elevated plus maze. Control mice were fed food lacking a supplement. Mice fed food supplemented with 5D and UDC spent less time in the closed arms of the maze (FIG. 14) and had lower active time in the closed arms of the maze than control mice (FIG. 15). Mice fed food supplemented with GDC did not spend significantly more time in closed arms than control mice.

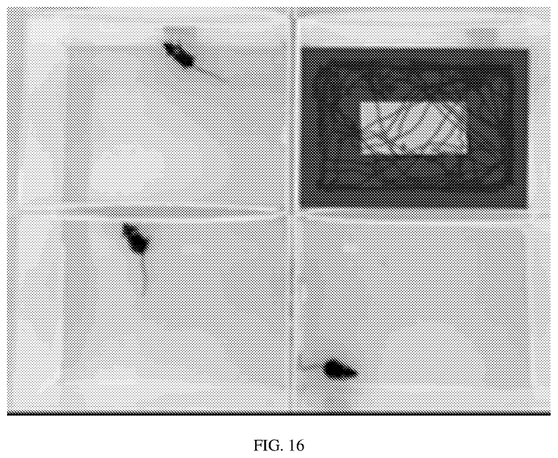

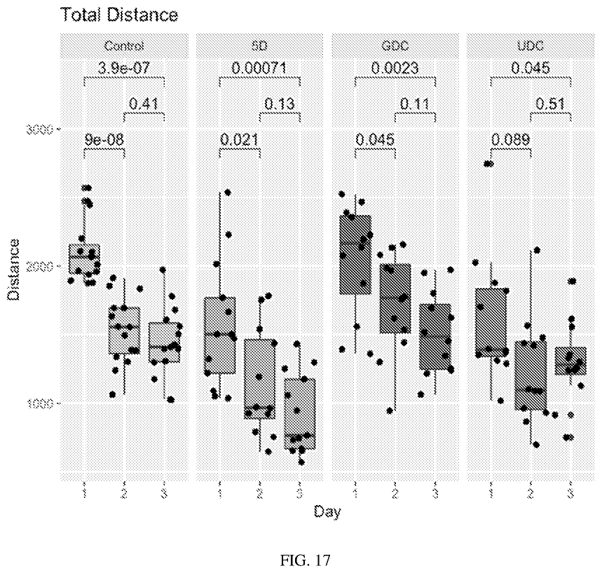

Habituation over was tested with the elevated plus maze (FIG. 16). Mice fed food supplemented with 5D or GDC showed habituation over three days. Specifically, mice fed food supplemented with 5D or GDC showed lower total distance traveled and exploratory activity over three days (FIG. 17). Mice fed food supplemented with a control metabolite did not habituate between days two and three. UDC did not decrease the total distance traveled on day three compared to day two, showing a lack of habituation. Significance was tested with an ANOVA test.

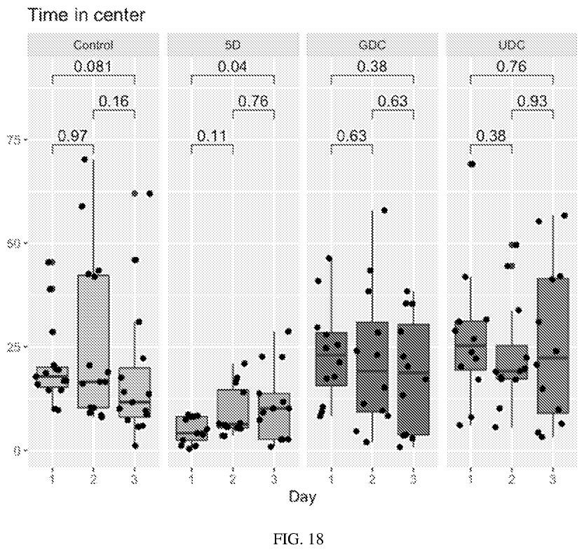

Habituation was measured by tracking the amount of time spent in the center of the chamber (FIG. 18). Mice fed food supplemented with 5D is significantly different from all other treatment groups, showing a trend for habituating to the center with an increase in time spent in the center of the chamber over time. Control mice did not habituate as they spent less time in the center of the chamber as time progressed.

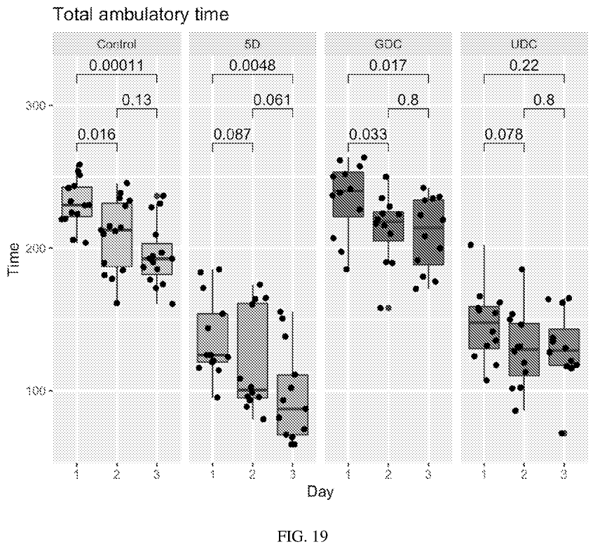

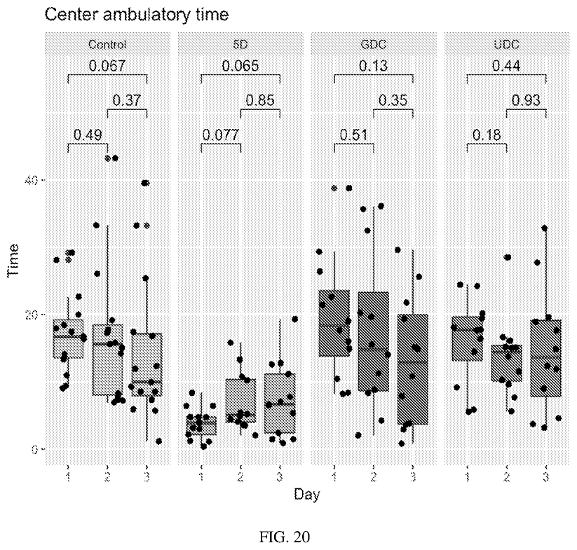

Active time was also measured as total ambulatory time and center ambulatory time. All mice showed less total ambulatory time over time (FIG. 19), but mice fed food supplemented with 5D showed exploratory behavior with an increase in center ambulatory time over time (FIG. 20).

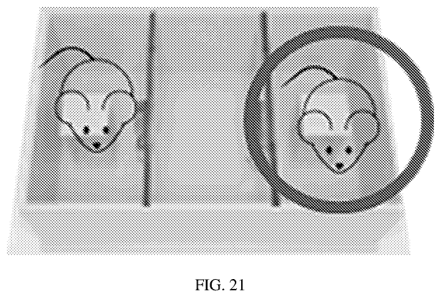

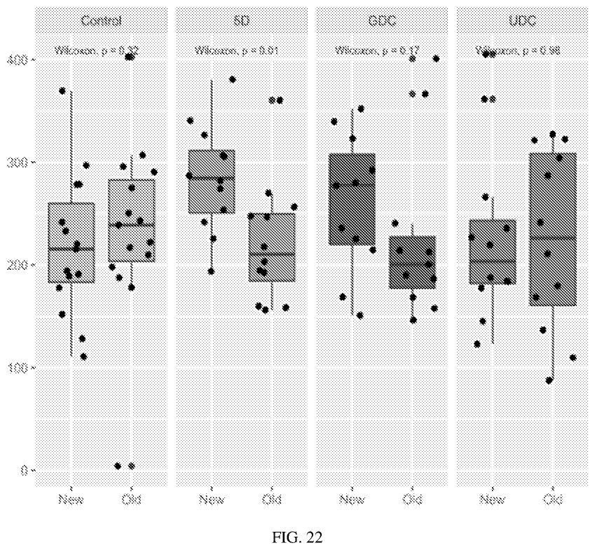

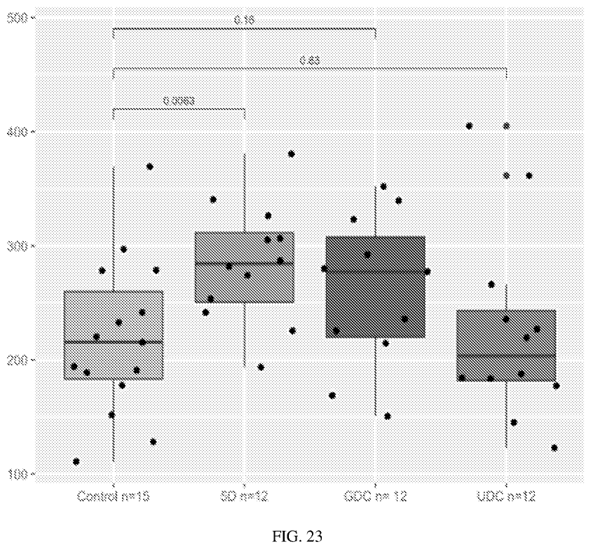

Additionally, the effect of supplementation with the metabolites on sociability was measured with a three-chamber sociability test (FIG. 21). This chamber tested for responses to social novelty. Mice fed food supplemented with 5D spent more time with a novel mouse (‘new’) than a mouse it had seen before (‘old’) (FIG. 22), and of the different treatments, spent the most time with a new mouse (FIG. 23).

TABLE 2
Behavioral assay summary.

| Mouse | EPM* | EPM | | Habituation | |
|---|---|---|---|---|---|
| Treatment | open | closed | Habituation | time in | |
| ↓ | arms | arms | exploration | center | Sociability |
| Control | NA | NA | No | No | No |
| 5D | No | Yes | Yes | Yes | Yes |
| GDC | No | No | Yes | No | No |
| UDC | No | Yes | No | No | No |

*EMP = Elevated Plus Maze

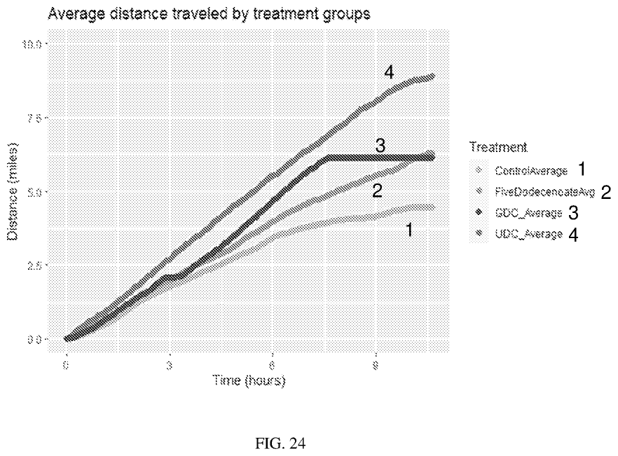

Lastly, mice fed with food supplemented with various metabolites were given a mouse wheel and distance traveled was tracked. All groups traveled significantly farther on average than the control group, although initial pace was the same (FIG. 24; Table 3). Also, night-time activity increased after administering metabolites for GDC, UDC and 5D.

TABLE 3
Comparison of average distance traveled by treatment group

| Treatment | Adjusted p value |
|---|---|
| 5D Average-Control Average | 0.0146608* |
| GDC Average-Control Average | 0.0000114* |
| UDC Average-Control Average | 0.00000000* |
| GDC Average-5D Average | 0.2761948 |
| UDC Average-5D Average | 0.00000000 |
| UDC Average-GDC Average | 0.0000141 |

*indicates statistical significance

### Example 4. Longitudinal Study of Stool-Associated Microbial Taxa in Sibling Pairs with and without Autism Spectrum Disorder

In this example, over 100 age-matched sibling pairs (between 2 to 8 years old) where one had an Autism ASD diagnosis and the other was developing typically (TD) were recruited (432 samples total). Stool samples were collected over four weeks, over 100 lifestyle and dietary variables were tracked, and behavior measures related to ASD symptoms were measured. 117 amplicon sequencing variants (ASVs) were identified that were significantly different in abundance between sibling pairs across all three time points, 11 of which were supported by at least two contrast methods. Additionally, dietary and lifestyle variables that differ significantly between cohorts were identified, and further linked to the ASVs they statistically relate to. Overall, dietary and lifestyle features were explanatory of ASD phenotype using logistic regression, however, global compositional microbiome features were not. Leveraging the longitudinal behavior questionnaires, 11 ASVs associated with changes in reported anxiety over time within and across all individuals were identified. Lastly, overall microbiome composition (beta-diversity) was associated with specific ASD-related behavioral characteristics.

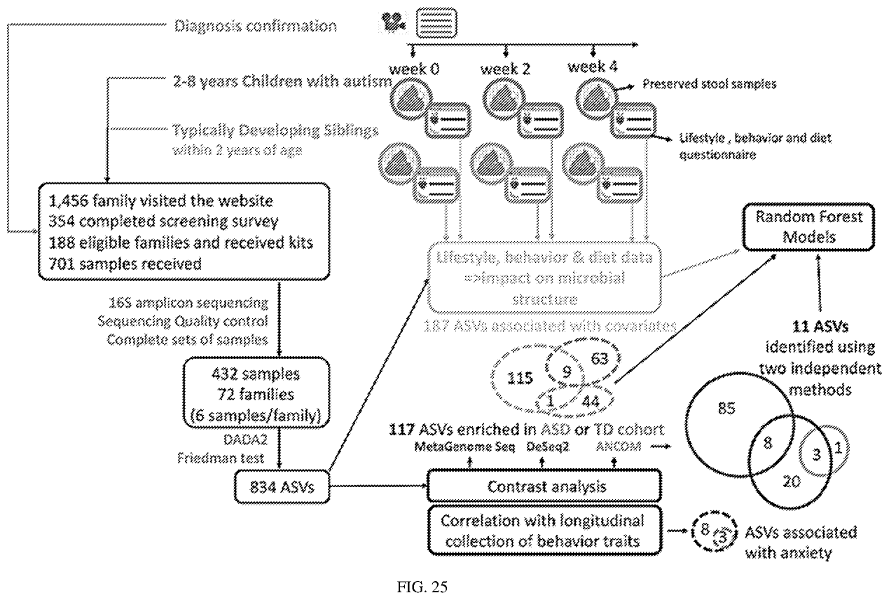

FIG. 25 shows an overall study design. Each sibling pair consisted of one ASD child and their respective TD sibling. Dietary, lifestyle, and other host variables were collected. The DADA2 pipeline was used to process the 16S V4 amplicon sequences. Samples from sibling pairs with ASD phenotypes unverified by parent reports or home videos were removed, leaving 432 samples. ASVs that significantly varied between timepoints in a Friedman test or were not present in 3% or more of the samples were removed. 117 ASVs were found to be significantly enriched in either the TD or ASD cohort. 11 of those ASVs were identified by more than one of the contrast methods shown above. Abundance counts of these 11 significant taxa are used as predictors in Random Forest Models.

Methods

Recruitment and Data Collection

Families with two siblings, one previously diagnosed with ASD by a health care provider and one typically developing were recruited. Children between 23 months to 8 years old, and siblings had to be within 2 years of each other were recruited. Dietary, lifestyle, demographic, and host health information were collected via an initial and bi-weekly questionnaires (at each collection time) for each individual. General dietary habits as well as recent dietary intake during the week prior was collected. In total, 1432 families visited the recruitment website.

Each sibling provided three stool samples, spaced two weeks apart. While 701 samples in total were received, sibling pairs with individuals that were younger than 23 months, were currently being breast-fed, or had ASD children that did not meet ASD criteria (See ASD Diagnosis Verification) were removed, leaving a total of 72 sibling pairs consisting of 432 samples from 144 different participants.

Autism Spectrum Disorder Diagnosis Verification

The Mobile Autism Risk Assessment (MARA), a parent reported behavioral questionnaire designed to screen children who are at high risk for ASD, was collected electronically from ASD participants.

Additionally, parents submitted a short video of their child with and child without ASD via encrypted file share to be rated for ASD symptoms on a set of 30 behavioral features. Scores across multiple raters were fed to previously published Machine Learning classifiers to predict ASD risk scores. By combining these risk scores with the parent-report screening tool (MARA), as well as parent-reported physician diagnosis, diagnosis was confirmed using majority rules consensus. Three children and their TD siblings were excluded for whom the consensus did not agree with original parent diagnosis.

Stool Collection and Storage

Every two weeks, caretakers of participants collected a sample using a provided toilet collection kit, and shipped it back at room temperature in preservation buffer (Norgen Biotek, ON, Canada). At the initial timepoint, caretakers also collected a second sample that was immediately frozen at home at −20° C., then shipped back overnight with two ice packs provided to the participants. Once received, stool samples were stored at −80° C. until processing.

DNA Extraction, Amplification, and Sequencing

Before DNA extraction, stool samples were thawed, pelleted, and supernatant was removed. DNA was extracted from the pelleted stool samples using the MagAttract PowerMicrobiome DNA/RNA Kit (Qiagen) on the KingFisher Flex 96 (ThermoFisher), following manufacturer's instructions. If DNA did not meet quality standards, an additional DNA clean-up procedure was performed with the Zymo ZR-96 DNA Clean-up kit. All samples were quantified via the Quant-iT PicoGreen dsDNA Assay Kit. The 16S rRNA V4 region was amplified with degenerate primers designed against conserved regions of the 16S rRNA V4 gene region, fused with Illumina adapters and indexing barcodes. The following primer sequences with adapters, pads and linkers were used:

Forward Primer:
(SEQ ID NO: 14)
AATGATACGGCGACCACCGAGATCTACACTATGGTAATTGTGTGYCAGC
MGCCGCGGTAA.
Reverse Primer:
(SEQ ID NO: 15)
CAAGCAGAAGACGGCATACGAGATXXXXXXXXXXXXXAGTCAGTCAGCC
GGACTACNVGGGTWTCTAAT (Where “XXXXXXXXXXXX”)
represents the indexing barcode).

PCR products were cleaned-up using AMPure XP beads (Beckman Coulter) and then quantified via the Quant-iT PicoGreen dsDNA Assay Kit (Invitrogen). Libraries were pooled, and paired-end sequencing (2×250 bps) was performed on an Illumina MiSeq using the MiSeq Reagent Kit v2 (500-cycles) and custom sequencing primers. An average of 157,103 reads per sample (with a min of 23,321 and max is 996,530) were obtained.

Sequence Processing, Filtering, and Taxonomic Annotation

Raw sequence reads were processed with DADA2 applying default settings for filtering, learning errors, dereplication, amplicon sequence variant (ASV) inference, and chimera removal. Truncation quality (truncQ) was set to 2. Ten nucleotides were trimmed from each terminus of each read. An average of 156, 246 reads per sample library remained after processing the raw reads. For strain level ASV assignment, ASVs were mapped to a strain database (StrainSelect, secondgenome.com/platform/data-analysis-tools/strainselect on the World Wide Web, version 2019 (SS19)) using USEARCH (usearch_global).

Statistical Analysis

Statistical analysis was performed using R version 3.6.2 using RStudio Server Pro 1.2.5033-1. The following packages were used: shiny 1.5.0, tibble 3.0.2, data.table 1.13.0, devtools 2.3.1, knitr 1.29, tidyr 1.1.0, reshape2 1.4.4, dplyr 1.0.0, ggplot2 3.3.2, pander 0.6.3, DT 0.14, gridExtra 2.3, adegraphics1.0-15, stats, smart 3.4-8, caret 6.0-86, randomforest 4.6-14, ROCR 1, 0-11, exactRankTests 0.8-31, nlme 3.1-148, compositions 2.0-0, ggpubr 0.4.0, vegan 2.5-6, MetagenomeSeq 1.28.2, DESeq2 1.26.0, biomformat 1.14.0, phyloseq 1.30.0 and sourced ANCOM 2.1 from github.com/FrederickHuangLin/ANCOM.git.

Normalization and Taxa Filtration

Before filtration, we attained a minimum read depth of 2.3*10⁴ reads and a maximum depth of 9.9*10⁵ reads. Taxa not present in at least 3% of the samples were removed. Taxa abundances were normalized using DESeq2 or Cumulative Sum Scaling (CSS) depending on the contrast analysis performed. Due to how DESeq2 normalized minimized intra-group variance within families more so than CSS, DESeq2 was used as the primary normalization in the gut-microbial community analysis.

In addition, taxa that significantly vary over time within the same individual were removed to increase the chance of identifying taxa directly related to core phenotype characteristics, rather than changes due to diet or season. A Friedman test was used to model ASV abundance as dependent on timepoint for each individual, and ASVs that were significantly related to timepoint (p<0.1) were removed. 64 ASVs were removed from DESeq normalized data, 78 ASVs were removed from CSS normalized data, and 72 ASVs were removed from unnormalized data.

Results

11 ASVs are Significantly Associated with the ASD Phenotype, as Determined by the Union of at Least Two Differential Analysis Methods

Out of 834 total ASVs (Amplicon Sequence Variants, assigned using DADA2), 117 were identified to be significantly different between the ASD and TD cohorts by at least one of the contrast analysis methods used after normalization and filtration (DESeq2, MetagenomeSeq, and ANCOM, see methods). Out of the 117 ASVs found to be significant across time points, 37 belonged to the Lachnospiraceae family. Oscillospiraceae and Bacteroidaceae were the second most represented families with 10 ASVs belonging to each of these families. 93 of the 117 ASVs were detected as significant by DESEQ2, 28 by MetagenomeSeq, and 4 by ANCOM. 45 ASVs were not associated with any lifestyle or dietary variables extracted from the questionnaires. Most notably, 11 ASVs were identified by at least 2 differential analysis methods.

Table 4 summarizes the 11 ASVs with overlapping detection between two contrast methods independently, and their lifestyle/dietary associations if applicable. Two of these were solely associated with the ASD cohort, and no other dietary or metadata co-variate: one from the genus Holdemania and one from the family Lachnospiraceae. Interestingly, the Blautia genus was represented in 3 of the 11 ASVs.

TABLE 4
Eleven ASVs significantly associated with the ASD or typically developing
cohorts by two independent contrasts methods.

| | | | | | Associated |
|---|---|---|---|---|---|
| Family | Genus | Species | Enrichment | Method | Variables |
| Bacteroidaceae | Bacteroides | thetaiotaomicron | ASD | DESEQ | ↓Seafood |
| | | | | 2, | consumption |
| | | | | MtgSeq | frequency |
| Lachnospiraceae | Blautia | unclassified | ASD | DESEQ | ↑Seafood |
| | | | | 2, | consumption |
| | | | | MtgSeq | frequency, |
| | | | | | ↑Dietary |
| | | | | | supplement*, |
| | | | | | ↑Multivitamin |
| Lachnospiraceae | Blautia_A | unclassified | ASD | DESEQ | ↓ Seafood |
| | | | | 2, | consumption |
| | | | | MtgSeq | frequency |
| Lachnospiraceae | unclassified | unclassified | ASD | DESEQ | None |
| | | | | 2, | |
| | | | | MtgSeq | |
| Oscillospiraceae | unclassified | unclassified | ASD | DESEQ | ↑GI symptoms |
| | | | | 2, | within 3 |
| | | | | MtgSeq | months* |
| Anaerovoracaceae | unclassified | unclassified | ASD | DESEQ | ↑Lactose |
| | | | | 2, | intolerance, |
| | | | | MtgSeq | ↑Dietary |
| | | | | | supplement*, |
| | | | | | ↓ Seafood |
| | | | | | consumption |
| | | | | | frequency |
| Erysipelotrichaceae | Holdemania | unclassified | ASD | DESEQ | None |
| | | | | 2, | |
| | | | | MtgSeq | |
| Christensenellaceae | Borkfalki | ceftriaxensis | ASD | DESEQ | ↑Dietary |
| | | | | 2, | supplement* |
| | | | | MtgSeq | |
| Streptococcaceae | Streptococcus | unclassified | TD | MtgSeq, | ↓Vegetable |
| | | | | ANCOM | consumption |
| | | | | | frequency (L), ↑ |
| | | | | | Dairy |
| | | | | | consumption |
| | | | | | (L)*, ↓Toilet- |
| | | | | | trained, ↓ |
| | | | | | Dietary |
| | | | | | restrictions*, |
| | | | | | ↓Multivitamin, |
| | | | | | ↓GI symptoms |
| | | | | | within 3 |
| | | | | | months* |
| Lachnospiraceae | Blautia_A | wexlerae | TD | MtgSeq, | ↓Vegetable |
| | | | | ANCOM | consumption |
| | | | | | frequency (L) ↑ |
| | | | | | Sugary food |
| | | | | | consumption |
| | | | | | (L), ↑ Dairy |
| | | | | | consumption |
| | | | | | (L)*, ↑Seafood |
| | | | | | consumption |
| | | | | | frequency, |
| | | | | | ↓Lactose |
| | | | | | intolerance, |
| | | | | | ↓Dietary |
| | | | | | supplement*, |
| | | | | | ↓GI symptoms |
| | | | | | within 3 |
| | | | | | months* |
| Pasteurellaceae | Haemophilus_ | unclassified | TD | MtgSeq, | ↓Pets in home, |
| | D | | | ANCOM | ↑Lactose |
| | | | | | intolerance, |
| | | | | | ↓Dietary |
| | | | | | restrictions*, |
| | | | | | ↓Multivitamin |

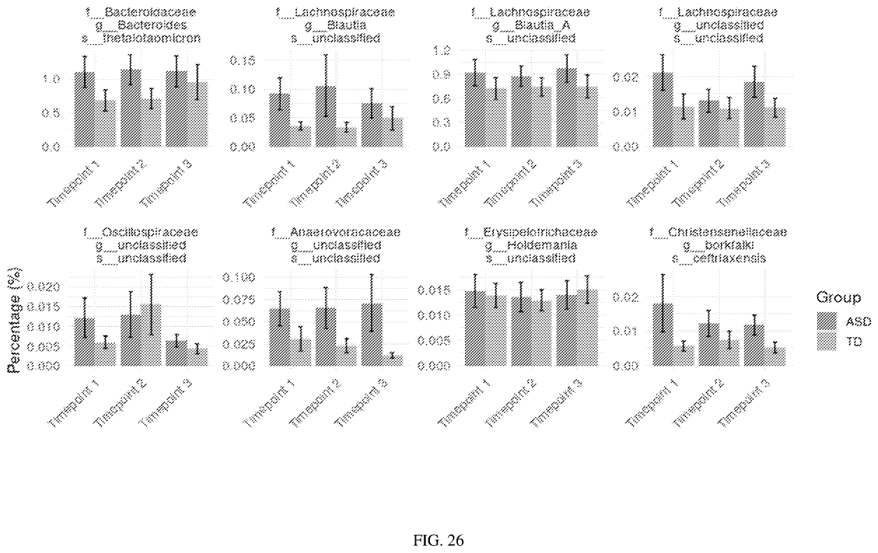

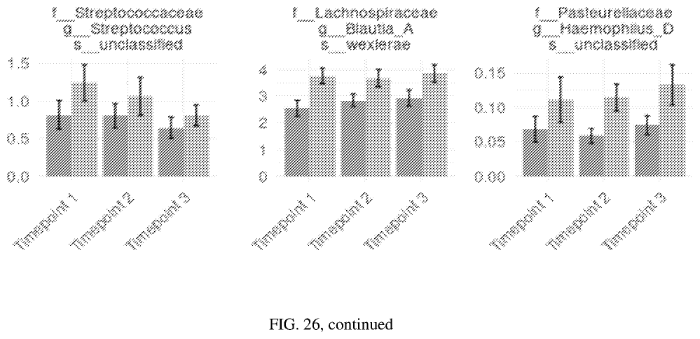

FIG. 26 shows total sum scaled abundance bar plots for the ASVs identified as significant by two methods. The ASV with the highest abundance among the 11 belonged to Blautia wexlerae with a relative abundance of almost 4% within the NT and around 2.5%-3% within the ASD cohort as shown in FIG. 26. ASVs from Bacteroides thetaiotaomicron and a different Blautia ASV were among the next highest abundances with values around 0.005%-0.01%.

Differential abundance testing was performed using all three contrast methods on ASV counts aggregated by annotated genus. Many of the differentially abundant ASVs from Table 1 were found to be members of differentially abundant genera, namely the genera Bacteroides, Borkfali, Haemophilus, and Streptococcus. Interestingly, the genus Veillonella was identified as increased in typically developing participants by all three analysis methods.

Demographics, Diet, and Lifestyle Differences Between Cohorts

331 diet and lifestyle variables were recorded for each individual participating. Unsurprisingly, as ASD has been found at a higher prevalence in males, 84.7% of the ASD cohort were male as compared to 52.7% of the TD cohort. There were no demographic differences between ASD and TD cohorts as siblings were exclusively documented as the same ethnicity.

A total of 14 of the 331 variables were significantly different between the ASD and TD cohorts. Categorical variables significant in chi-squared tests between cohorts are shown in Table 5 as well as cohort age and C-section birth status. Notably, bowel function and GI symptoms were observed significantly more often in ASD participants, as were special dietary regimes and dietary supplementation (adjusted p<0.05 in wilcoxon rank sum tests or two-way repeated anovas). Six of these variables were also associated with microbial community dissimilarities using the Bray-Curtis distance metric tested by PERMANOVA. These variables were “dietary restriction”, “dietary supplementation”, “GI symptoms within 3 months”, “GI issues this week”, “habitual fruit consumption”, and “last 2 weeks dairy consumption”.

Dietary/Lifestyle, but not Global Microbiome Compositional Features, Explain ASD Phenotype

To assess the overall associations between lifestyle, microbial factors, and ASD phenotype, logistic regression using different feature sets were used as follows: 1) Basic (Age+Sex), 2) Basic+lifestyle/dietary variables, 3) Basic+microbiome features, 4) Basic+lifestyle/diet variables+microbiome features. Microbiome features were calculated as scores along a principal coordinate ordination using Bray-Curtis distance. Additionally, null models were created by replacing features with uniformly randomly distributed noise.

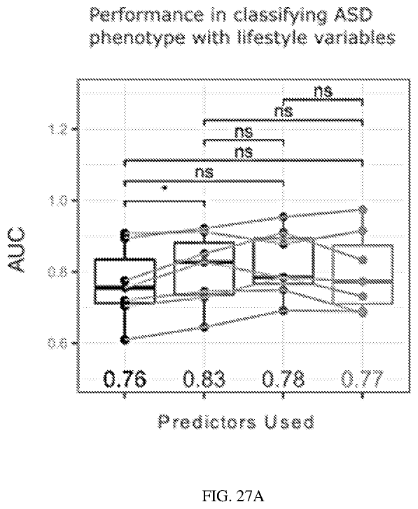

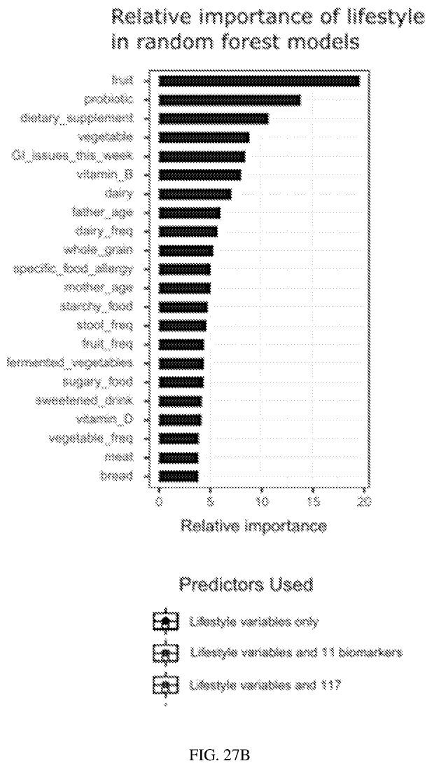

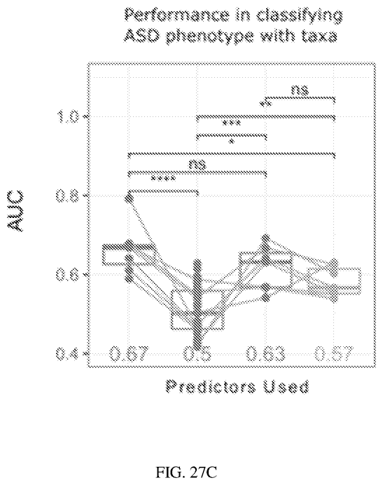

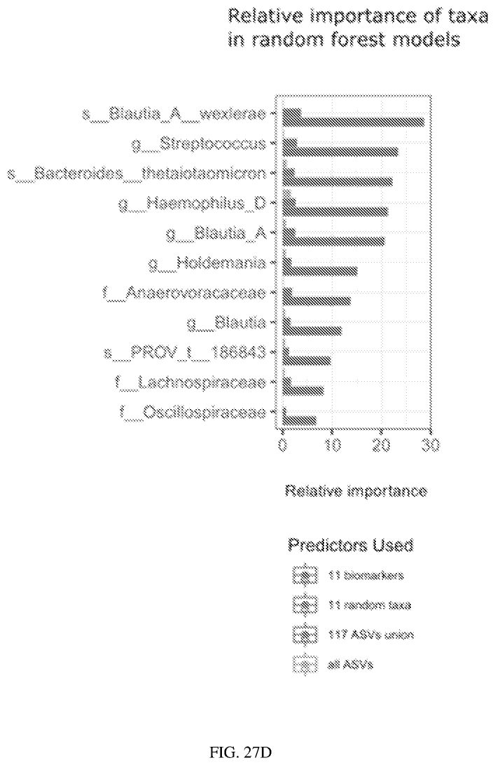

Inclusion of lifestyle/dietary variables, but not of microbiome features, significantly improved the explanatory power of a model over basic features (FIG. 27). Microbiome features did explain phenotype significantly more accurately than random noise variables. A combination of lifestyle and microbiome features did not significantly improve performance over lifestyle features.

TABLE 5
ASD and TD comparison of demographic information
and significant lifestyle variables.

| | | | | Associated |
|---|---|---|---|---|
| | | | | with Microbial |
| | ASD | TD | | Community |
| Per Child | (n = 72) | (n = 72) | q-value | Structure |
| Probiotic consumption | | | | |
| Never | 15/72 | 38/72 | 6.94E−04 | No |
| Rarely | 9/72 | 11/72 | | |
| Occasionally | 3/72 | 1/72 | | |
| Regularly | 6/72 | 2/72 | | |
| Weekly | 8/72 | 6/72 | | |
| Several times weekly | 11/72 | 9/72 | | |
| Daily | 19/72 | 9/72 | | |
| Vitamin B | | | | |
| supplementation | | | | |
| Never | 40/72 | 57/72 | 1.09E−02 | No |
| Rarely | 2/72 | 7/72 | | |
| Occasionally | 9/72 | 1/72 | | |
| Regularly | 3/72 | 1/72 | | |
| Several times weekly | 3/72 | 1/72 | | |
| Daily | 15/72 | 5/72 | | |
| Vitamin D | | | | |
| consumption | | | | |
| Never | 35/72 | 47/72 | 3.25E−02 | No |
| Rarely | 1/72 | 11/72 | | |
| Occasionally | 3/72 | 0/72 | | |
| Regularly/Weekly | 9/72 | 4/72 | | |
| Several times weekly | 6/72 | 4/72 | | |
| Daily | 1/72 | 6/72 | | |
| Dietary supplement² | | | | |
| True | 37/61 | 14/61 | 1.40E−03 | Yes (q = 0.002) |
| False | 24/61 | 47/61 | | R² = 0.007 |
| Dietary restrictions | | | | |
| True | 27/72 | 8/72 | 1.08E−02 | Yes (q = 0.001) |
| False | 45/72 | 64/72 | | R² = 0.008 |
| Functional bowel | | | | |
| finding¹ | | | | |
| Tends to have diarrhea | 19/72 | 7/71 | 7.47E−03 | No |
| Tends to have | 13/72 | 3/71 | | |
| constipation | 40/72 | 61/71 | | |
| Tends to have normal | | | | |
| BM | | | | |
| GI symptoms within 3 | | | | |
| months | | | | |
| True | 36/72 | 64/72 | 2.80E−05 | Yes (q = 0.004) |
| False | 36/72 | 8/72 | | R² = 0.003 |
| Biological sex | | | | |
| Male | 61/72 | 38/72 | 1.91E−03 | No |
| Female | 11/72 | 34/72 | | |
| Age | | | | |
| Mean | 5.39 | 4.90 | 0.15 | No |
| Standard deviation | 1.42 | 2.43 | | |
| C-section birth | | | | |
| True | 27/72 | 27/72 | 1.00 | No |
| False | 45/72 | 45/72 | | |
| Fruit Consumption | | | | |
| Never | 25/72 | 35/72 | 3.26E−02 | Yes (q = 0.002) |
| Rarely | 9/72 | 0/72 | | R² = 0.027 |
| Weekly | 11/72 | 2/72 | | |
| Several times weekly | 21/72 | 23/72 | | |
| Daily | 6/72 | 12/72 | | |
| | | | | Associated |
| | | | | with Microbial |
| | ASD | TD | | Community |
| Per Timepoint | (n = 216) | (n = 216) | | Structure |
| Dairy Consumption*^{M} | | | | |
| Never/less than once a | 90/203 | 36/202 | 3.75E−02 | Yes (q = 0.001) |
| week | | | | |
| 3-4 meals per week | 33/203 | 45/202 | | R² = 0.009 |
| 7-10 meals per week | 42/203 | 76/202 | | |
| Almost every meal | 38/203 | 45/202 | | |
| Recent Anxiety^{M} | | | | |
| No elevated anxiety | 151/203 | 185/202 | 1.98E−02 | No |
| Somewhat elevated | 34/203 | 13/202 | | |
| anxiety | | | | |
| Elevated anxiety | 18/203 | 4/202 | | |
| GI issues this week | | | | |
| True | 66/216 | 9/216 | 1.02E−11 | Yes (q = 0.003) |
| False | 150/216 | 207/216 | | R² = 0.005 |
| Other symptoms this | | | | |
| week³ | | | | |
| True | 4/214 | 23/214 | 2.76E−03 | No |
| False | 210/214 | 191/214 | | |

¹Missing one TD response.
²Missing 11 TD and ASD responses resulting in an n of 61 out of 72 per cohort.
³Missing responses for two timepoints in both ASD and TD.
^{M}Per timepoint dietary consumption and anxiety measures had 13 missing ASD responses and 14 TD responses resulting in an n of 203 or 202.
*This variable was significant in per timepoint (question answered every 2 weeks) and during initial assessment.
The q-value column is the adjusted p-value from wilcoxon-ranked sum tests, chi-squared tests, or two-way repeated measures anovas based on the category of variable.
Age and C-section birth status are the only two variables included in this table that were not significantly different between cohorts.
Age is listed in years.
Racial demographic information and household information not included as they are the same across cohorts for each sibling pair.
All participants were from the United States.

Because highly correlated variables are difficult to distinguish using regression models, the Pearson correlation matrix between all significant lifestyle variables and other lifestyle variables was calculated (FIG. 27B). High bread, multivitamin, fermented vegetable, and olive oil consumption, along with GI distress and non-celiac sensitivity, are significant predictors of ASD. Home prepared meals, as opposed to ready-to-eat meals, were inversely correlated with both ASD phenotype and GI distress.

While the major axes of variation within the gut microbiome did not present additional explanatory power on top of age and sex, some of the axes were statistically significantly related to phenotype. Correlations between axes coordinates and lifestyle variables are found in FIG. 27C. Most notably, a sample's position along the axes of highest variation (axis1) was associated with the TD phenotype, and scores along this axis correlated with vegetable, fruit, and fat/oil consumption, in addition to meats, seafood, and in general eating home prepared meals.

Some principal component axes, while not obviously correlated with any lifestyle characteristic, were enriched for the 8 biomarkers associated with ASD or the 3 biomarkers associated with TD (FIG. 27, Table 4). A modified gene set enrichment analysis where a set was considered the 8 ASD or 3 TD biomarkers revealed that scores for biomarkers along particular significant axes were more skewed than would be expected by random chance (gsea p<0.05) (FIG. 27D).

11 Taxa Correlate with Anxiety Scores within and Across Individuals

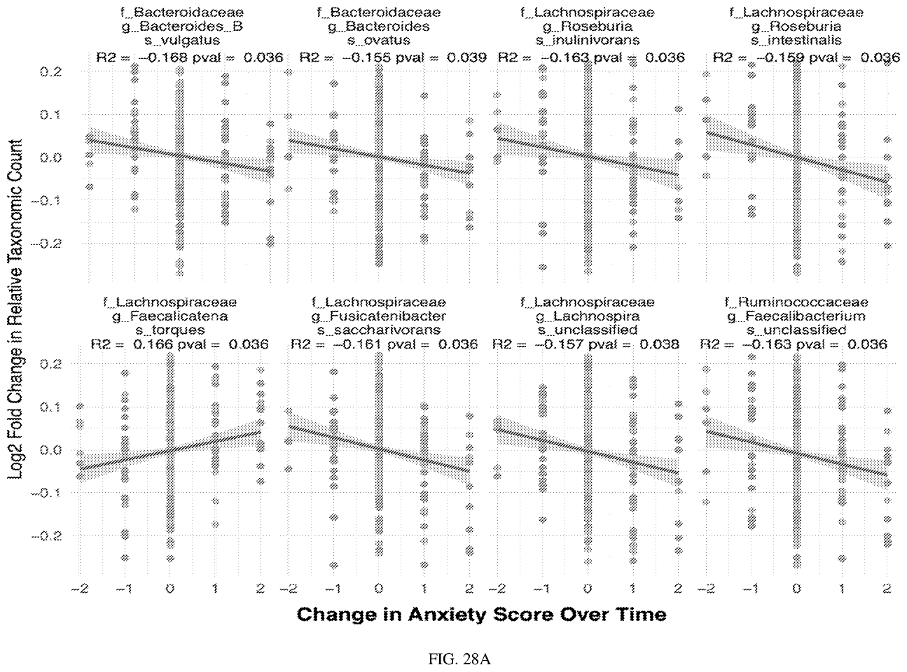

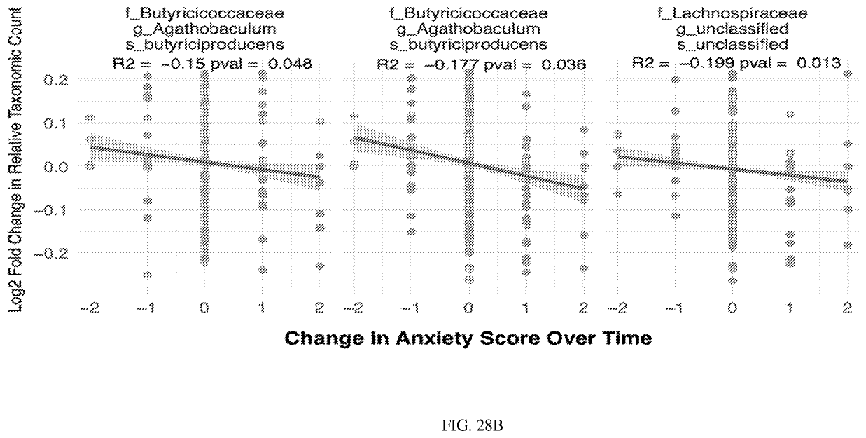

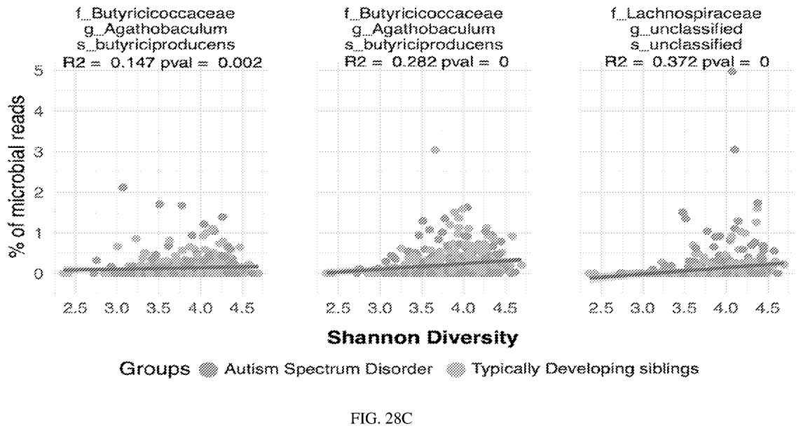

Anxiety in the last 2 weeks before each sample collection was reported by caretakers on a scale of “No elevated anxiety”, “Somewhat elevated”, and “elevated” (0, 1, 2). This metric was used to measure changes in anxiety within the same individual across time, allowing full leverage the longitudinal nature of the data to identify specific ASVs associated with reported anxiety. 10 ASVs significantly negatively correlated and 1 ASV positively correlated with increasing anxiety (FIGS. 28A-28B, Table 6). Two ASVs from the species A. butyriciproducens, a butyrate producing bacteria, were both negatively correlated with anxiety. Six of the 10 ASVs negatively correlated with anxiety were members of the Lachnospiraceae family. Three of the ASVs found correlated with anxiety in the full cohort were similarly correlated with anxiety when considering only the ASD samples (FIG. 28B)

Multiple diversity metrics (Chao1, Shannon, FaithPD) correlated with ASD severity score (MARA) and age, however, diversity was not significantly different between ASD and TD cohorts.

TABLE 6
11 Taxa Correlated with Anxiety Scores Within and Across
Individuals

| SEQ ID | | | | | Padj | r2 |
|---|---|---|---|---|---|---|
| NO: | Sequence | Family | Genus | Species | values | values |
| SEQ ID | ACAAG | Ruminoco | Faecaliba | spp. | 0.0354 | −0.1632 |
| NO: 3 | CGTTGT | ccaceae | cterium | | | |
| | CCGGA | | | | | |
| | ATTACT | | | | | |
| | GGGTGT | | | | | |
| | AAAGG | | | | | |
| | GAGCG | | | | | |
| | CAGGC | | | | | |
| | GGGAG | | | | | |
| SEQ ID | CCGAGC | Bacteroid | Bacteroides | vulgatus | 0.0354 | −0.1679 |
| NO: 4 | GTTATC | aceae | | | | |
| | CGGATT | | | | | |
| | TATTGG | | | | | |
| | GTTTAA | | | | | |
| | AGGGAG | | | | | |
| | CGTAGA | | | | | |
| | TGGATG | | | | | |
| | TTTAAG | | | | | |
| SEQ ID | CCGAGCGTTATCCG | Bacteroidaceae | Bacteroides | ovatus | 0.0389 | −0.1547 |
| NO: 5 | GATTTATTGGGTTT | | | | | |
| | AAAGGGAGCGTAG | | | | | |
| | GTGGATTGTTAAGT | | | | | |
| | CAGTTGTGAAAGTT | | | | | |
| | TGCGGCTCAACCGT | | | | | |
| | AAAATTGCAGTTG | | | | | |
| | AAACTGGCAGTCTT | | | | | |
| | GAGTACAGTAGAG | | | | | |
| SEQ ID | GCAAGCGTTATCCG | Lachno- | Roseburia | inulinivorans | 0.0354 | −0.1634 |
| NO: 6 | GATTTACTGGGTGT | spiraceae | | | | |
| | AAAGGGAGCGCAG | | | | | |
| | GCGGAAGGCTAAG | | | | | |
| | TCTGATGTGAAAGC | | | | | |
| | CCGGGGCTCAACC | | | | | |
| | CCGGTACTGCATTG | | | | | |
| | GAAACTGGTCATCT | | | | | |
| | AGAGTGTCGGAGG | | | | | |
| SEQ ID | GCAAGCGTTATCCG | Lachno- | Roseburia | intestinalis | 0.0354 | −0.1593 |
| NO: 7 | GATTTACTGGGTGT | spiraceae | | | | |
| | AAAGGGAGCGCAG | | | | | |
| | GCGGTACGGCAAG | | | | | |
| | TCTGATGTGAAAGC | | | | | |
| | CCGGGGCTCAACC | | | | | |
| | CCGGTACTGCATTG | | | | | |
| | GAAACTGTCGGAC | | | | | |
| | TAGAGTGTCGGAG | | | | | |
| SEQ ID | GCAAGCGTTATCCG | Lachno- | Faecali- | torques | 0.0354 | 0.1662 |
| NO: 8 | GATTTACTGGGTGT | spiraceae | catena | | | |
| | AAAGGGAGCGTAG | | | | | |
| | ACGGATGGGCAAG | | | | | |
| | TCTGATGTGAAAAC | | | | | |
| | CCGGGGCTCAACC | | | | | |
| | CCGGGACTGCATTG | | | | | |
| | GAAACTGTTCATCT | | | | | |
| | AGAGTGCTGGAGA | | | | | |
| SEQ ID | GCAAGCGTTATCCG | Lachno- | Fusicateni- | sacchari- | 0.0354 | −0.1610 |
| NO: 9 | GATTTACTGGGTGT | spiraceae | bacter | vorans | | |
| | AAAGGGAGCGTAG | | | | | |
| | ACGGCAAGGCAAG | | | | | |
| | TCTGATGTGAAAAC | | | | | |
| | CCAGGGCTTAACCC | | | | | |
| | TGGGACTGCATTGG | | | | | |
| | AAACTGTCTGGCTC | | | | | |
| | GAGTGCCGGAGAG | | | | | |
| SEQ ID | GCAAGCGTTATCCG | Lachno- | Lachnospira | spp. | 0.0373 | −0.1569 |
| NO: 10 | GATTTACTGGGTGT | spiraceae | | | | |
| | AAAGGGAGTGTAG | | | | | |
| | GTGGCCATGCAAG | | | | | |
| | TCAGAAGTGAAAA | | | | | |
| | TCCGGGGCTCAACC | | | | | |
| | CCGGAACTGCTTTT | | | | | |
| | GAAACTGTAAGGC | | | | | |
| | TAGAGTGCAGGAG | | | | | |
| SEQ ID | GCAAGCGTTATCCG | Lachno- | unclassified | spp. | 0.0129 | −0.1990 |
| NO: 11 | GATTTACTGGGTGT | spiraceae | | | | |
| | AAAGGGAGTGTAG | | | | | |
| | GTGGTATCACAAGT | | | | | |
| | CAGAAGTGAAAGC | | | | | |
| | CCGGGGCTCAACC | | | | | |
| | CCGGGACTGCTTTT | | | | | |
| | GAAACTGTGGAAC | | | | | |
| | TGGAGTGCAGGAG | | | | | |
| SEQ ID | GCAAGCGTTATCCG | Butyr- | Agatho- | butyr- | 0.0479 | −0.1502 |
| NO: 12 | GATTTACTGGGTGT | icicoccaceae | baculum | iciproducens | | |
| | AAAGGGCGCGCAG | | | | | |
| | GCGGGCCGGCAAG | | | | | |
| | TTGGAAGTGAAAT | | | | | |
| | CTATGGGCTTAACC | | | | | |
| | CATAAACTGCTTTC | | | | | |
| | AAAACTGCTGGTCT | | | | | |
| | TGAGTGATGGAGA | | | | | |
| SEQ ID | GCAAGCGTTATCCG | Butyr- | Agatho- | butyr- | 0.0354 | −0.1774 |
| NO: 13 | GATTTACTGGGTGT | icicoccaceae | baculum | iciproducens | | |
| | AAAGGGCGCGCAG | | | | | |
| | GCGGGCCGGTAAG | | | | | |
| | TTGGAAGTGAAAT | | | | | |
| | CTATGGGCTTAACC | | | | | |
| | CATAAACTGCTTTC | | | | | |
| | AAAACTGCTGGTCT | | | | | |
| | TGAGTGATGGAGA | | | | | |

Ten Behavioral Variables are Associated with the Microbial Structure within the ASD Cohort

Out of the 14 behavioral questions within the Mobile Autism Risk Assessment (MARA) collected in the ASD cohort, 10 were significantly associated with gut microbiome composition (Table 7). Constrained PCOAs using Bray-Curtis distances of DESEQ2 normalized counts were created for each of the significant behavioral variables.

TABLE 7
Significant Behavioral Variables Associated with
Overall Microbial Structure in the ASD cohort.

| | Factor | Total | | |
|---|---|---|---|---|
| Variable | class | samples | R² | q-value |
| Childhood behavioral | numeric | 189 | 0.016 | 0.005 |
| development finding | | | | |
| Plays imaginatively | numeric | 216 | 0.015 | 0.005 |
| with others | | | | |
| Plays in a group with others | numeric | 216 | 0.011 | 0.005 |
| Language ability and use | numeric | 216 | 0.011 | 0.005 |
| Sleep pattern finding | numeric | 216 | 0.01 | 0.005 |
| Eye contact finding | numeric | 216 | 0.01 | 0.005 |
| Repetitive motion | numeric | 216 | 0.009 | 0.011 |
| Response to typical sounds | numeric | 216 | 0.009 | 0.016 |
| Imitation behavior | numeric | 216 | 0.008 | 0.028 |
| Picks up objects to | numeric | 216 | 0.008 | 0.033 |
| show others | | | | |

Variables listed above have q-values < 0.05.

### Example 5. Impact of 5D Supplementation on the Gut Microbiota

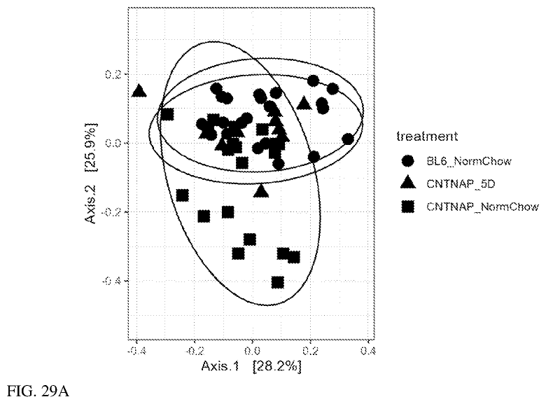

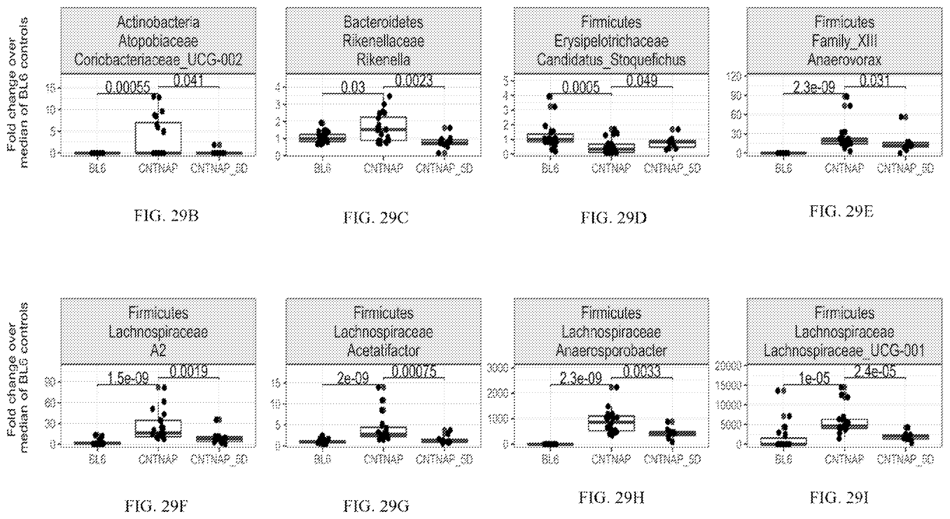

In this example, oral supplementation with 5D shifts the taxonomic composition of CNTNAP2 mice (a mice model of autism, where the gene contactin associated protein-like 2 CNTNAP2 has been knockout, a model commonly used in autism because this gene is expressed in human brain regions related to the disorder and with evidence for both rare and common variation contributing to ASD) towards the composition of C57BL/6 wild types (FIG. 29A). Ten genera of bacteria were identified that were observed to be increased in CNTNAP2 mice compared to C57BL/6, and then significantly decreased by supplementation with 5D by a Wilcoxon rank sum test (FIGS. 29B-29N). Genera under the family Lachnospiraceae and Ruminococcaceae were consistently seen increased in CNTNAP2 mice and decreased with 5D supplementation. At a species level, a single species, Romboutsia ilealis, was found at very low levels in C57BL/6 and CNTNAP2 mice fed standard chow, but found at very high levels in some CNTNAP2 mice fed 5D.

FIGS. 29A-29N show that taxonomic composition in the colons differs between C57BL/6, CNTNAP2, and CNTNAP2 mice supplemented with 5D. FIG. 29A shows Principal Coordinate analysis (PCoA) plot of Bray-Curtis distances between treatments. The results showed increased similarity between C57BL/6 and CNTNAP2 mice supplemented with 5D as compared between C57BL/6 and CNTNAP2 mice fed standard chow. P-values were determined using permanova and beta-dispersion tests. FIGS. 29B-29N show results of differential abundance test of ASVs aggregated at the genus level. The results showed that members of the Lachnospiraceae and Ruminococcaceae families were most frequently increased in CNTNAP2 mice fed standard chow and decreased by 5D supplementation. P-values were determined using Wilcoxon rank sum test, values were reported as fold change of median of C57BL/6 controls.

### Example 6. 5-Dodecenoate Supplementation Shifted the Lipid Profiles in Liver, Plasma, and Frontal Cortex

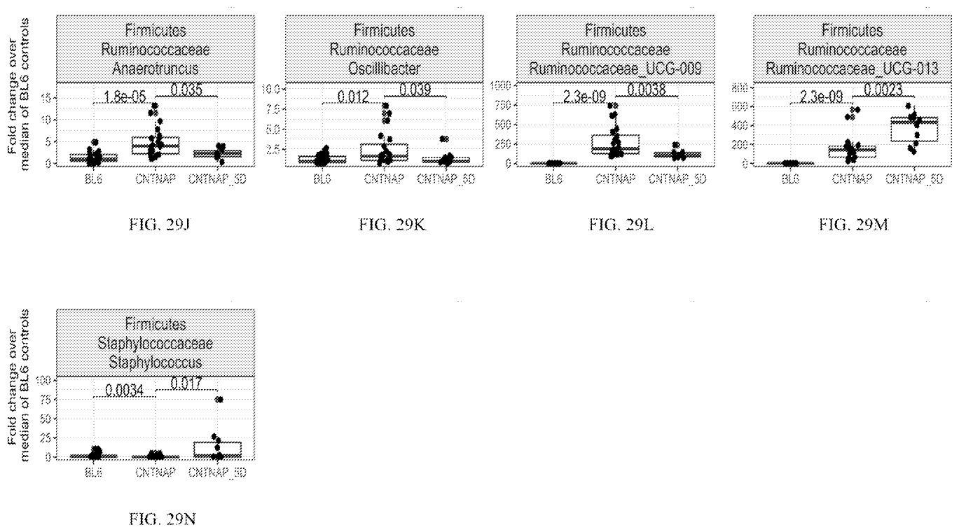

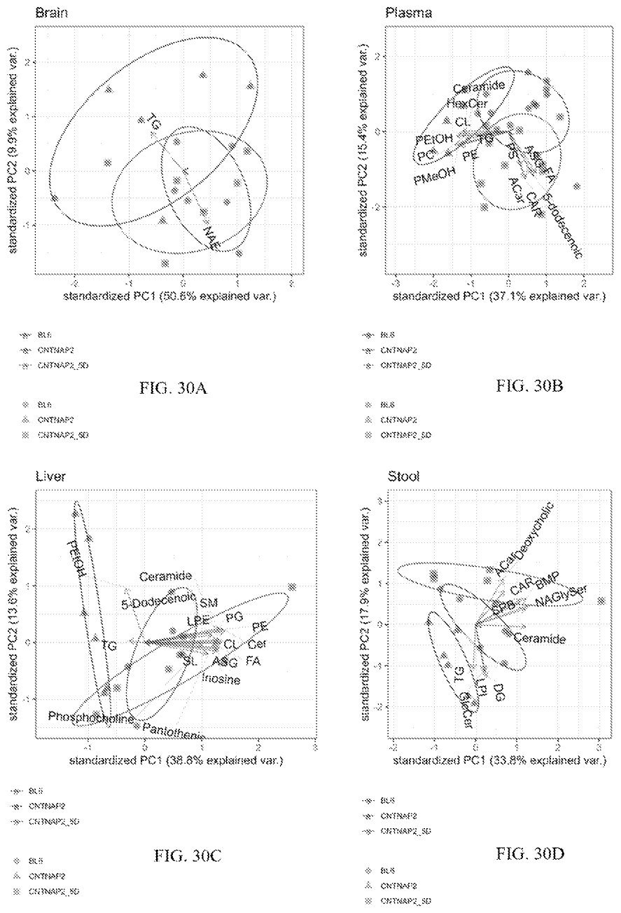

In this example, CNTNAP2 mice were observed to have significantly different lipid profiles from C57BL/6 controls at baseline in brain, plasma, and liver, and stool (PERMANOVA p=0.044). 5D supplementation was observed to significantly shift CNTNAP2 mouse lipid profiles in plasma and liver, and tended to shift brain and stool profiles, though this shift was not statistically significant. The result showed that triglycerides are the lipid factor most consistently related to the observed shifts between conditions across every tissue measured and shown in FIGS. 30A-30D.

FIGS. 30A-30D show that lipidomic profiles in brain (FIG. 30A), plasma (FIG. 30B), liver (FIG. 30C), and stool (FIG. 30D) shifted both with CNTNAP2 mutant and 5D supplementation. Triglycerides were identified as influential in every tissue measured. Lipids were first aggregated into categories (e.g. triglycerides, phosphocholines), then PCA ordination was applied. Influence on PCA plot of individual lipid classes that significantly differ between conditions are shown as arrows (significance cutoff rank sum test p<0.01). P-values determined from permanova test between treatments are shown as annotations.

### Example 7. Elevated Triglycerides in all Tissues of CNTNAP2 Mice were Rescued by 5D Supplementation

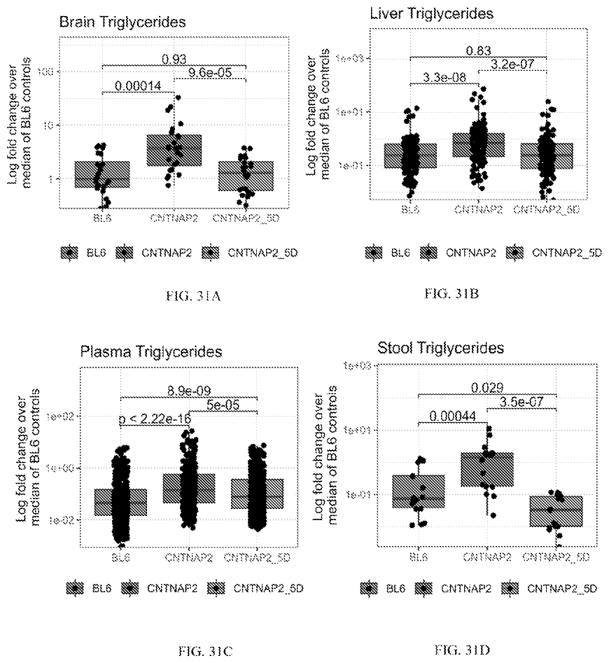

In this example, it was determined that triglyceride levels were significantly elevated in all four tissue fractions of the CNTNAP2 control mice (FIGS. 31A-31D). The results showed that 5D dietary supplementation decreased the levels significantly in each of the tissues back to levels consistent with the C57BL/6 controls.

FIGS. 31A-31D show that triglyceride levels in brain (FIG. 31A), liver (FIG. 31B), plasma (FIG. 31C), and stool (FIG. 31D) were elevated in CNTNAP2 mice and reduced significantly by 5D supplementation. Intensities were normalized using CUDA calibration and were reported as log fold change of median of C57BL/6 wild type controls. P-values were derived from Wilcox rank sum test.

### Example 8. 5-Dodecenoate Supplementation Modulated Gene Pathways Associated with Autism in Transcriptomics of the Frontal Cortex

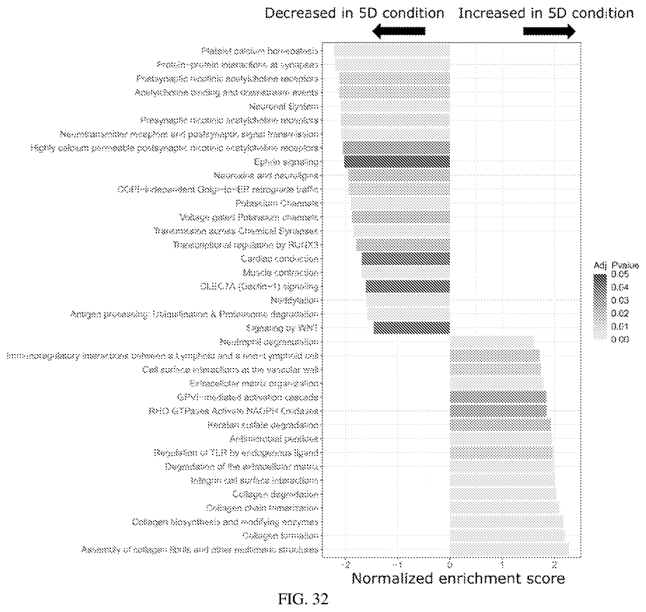

In this example, to further investigate 5D's effect on neuronal function, RNA-sequencing was performed on the frontal cortex region of CNTNAP2 mice supplemented with 5D after 3 weeks. Differential analysis revealed 1438 genes that were significantly modulated in the 5D fed condition (p<0.05), and gene set enrichment analysis identified functional pathways significantly affected by those differences (FIG. 32, Table 8). The results showed that 5D supplementation decreased expression of synapse-dependent pathways related to postsynaptic signal transmission, transmission across chemical synapses, potassium channels, and presynaptic nicotinic acetylcholine receptors. The results also showed that 5D supplementation increased the expression of pathways such as collagen formation, biosynthesis and degradation, integrin cell surface interactions, extracellular matrix organization and degradation, all of which broadly relate to cell structure and interaction. The results also showed that the supplementation increased expression of pathways that relate to innate immunity such as neutrophil degranulation, antimicrobial peptides, regulation of Toll-like receptors (TLR) by endogenous ligands, and immunoregulatory interactions (FIG. 32).

FIG. 32 also shows that 5D supplementation affects gene transcription in the frontal cortex. Gene set enrichment analysis on RNA-seq data from the frontal cortex region showed that synapse dependent functions, specifically nicotinic acetylcholine receptors, were downregulated by 5D supplementation while collagen and cell structure gene pathways were upregulated. Bar size depicts enrichment score normalized by gene set size, and color depicts fdr corrected p-value as determined by gene set enrichment analysis. The results showed that positive enrichment scores increased with 5D supplementation. (CNTNAP-NormC: n=5, CNTNAP-5D: n=9)

TABLE 8
Gene set enrichment analysis on RNA-seq data from frontal cortex of CNTNAP2
mice fed normal chow (n = 5) and supplemented with 5D (n = 9).

| | | normalized | | | |
|---|---|---|---|---|---|
| | | enrichment | | | |
| ID | Description | score | p.adjust | coverage | core enrichment genes |
| R- | Platelet calcium | −2.258 | 0.0039 | 0.44 | Itpr1, P2rx6, P2rx7, Slc8a3, |
| MMU- | homeostasis | | | | Calm3, P2rx3, Atp2a3, |
| 418360 | | | | | Slc8a2, Atp2b4, P2rx5, |
| | | | | | Atp2b2, P2rx2 |
| R- | Protein-protein | −2.181 | 0.0003 | 0.52 | Ntrk3, Gria4, Nlgn2, Lrfn4, |
| MMU- | interactions at | | | | Dlgap2, Slitrk5, Nrxn1, |
| 6794362 | synapses | | | | Lrrtm2, Grin2a, Grm1, |
| | | | | | Homer2, Lrrc4b, Lrfn3, |
| | | | | | Dlg4, Epb4ll1, Ppfia3, |
| | | | | | Lrfn2, Dlgap1, Slitrk4, |
| | | | | | Il1rapl2, Ptprs, Cask, |
| | | | | | Dlgap3, Grm5, Dlg2, |
| | | | | | Nlgn3, Lrfn1, Slitrk1, |
| | | | | | Flot1, Nlgn1, Shank2, |
| | | | | | Shank1, Shank3, Ptprf, |
| | | | | | Grin2b |
| R- | Acetylcholine | −2.154 | 0.0132 | 0.42 | Chrna3, Chrnb4, Chrna7, |
| MMU- | binding and | | | | Chrna4, Chrnb3, Chrna6 |
| 181431 | downstream events | | | | |
| R- | Postsynaptic | −2.154 | 0.0132 | 0.42 | Chrna3, Chrnb4, Chrna7, |
| MMU- | nicotinic | | | | Chrna4, Chrnb3, Chrna6 |
| 622327 | acetylcholine | | | | |
| | receptors | | | | |
| R- | Presynaptic | −2.107 | 0.0079 | 0.4 | Chrna3, Chrnb4, Chrna4, |
| MMU- | nicotinic | | | | Chrnb3, Chrna6 |
| 622323 | acetylcholine | | | | |
| | receptors | | | | |
| R- | Highly calcium | −2.095 | 0.0232 | 0.45 | Chrna3, Chrnb4, Chrna7, |
| MMU- | permeable | | | | Chrna4, Chrnb3, Chrna6 |
| 629594 | postsynaptic | | | | |
| | nicotinic | | | | |
| | acetylcholine | | | | |
| | receptors | | | | |
| R- | Neurotransmitter | −2.033 | 0 | 0.44 | Gng3, Gabra1, Gabbr1, |
| MMU- | receptors and | | | | Prkaca, Gria4, Gna1, |
| 112314 | postsynaptic signal | | | | Adcy5, Kcnj9, Chrna5, |
| | transmission | | | | Camk2d, Kcnj15, Gng2, |
| | | | | | Prkar1b, Prkacb, Gabbr2, |
| | | | | | Grin2a, Prkar2b, Gabrr2, |
| | | | | | Gnb2, Adcy8, Grik5, |
| | | | | | Akap5, Prkar1a, Grik4, |
| | | | | | Dlg4, Grin3a, Epb4ll1, |
| | | | | | Cacng3, Plcb1, Chrna3, |
| | | | | | Chrnb4, Camkk2, Nsf, |
| | | | | | Rps6ka2, Gabra3, Grip1, |
| | | | | | Dlg2, Ap2m1, Ap2a2, |
| | | | | | Calm3, Gng13, Camk2a, |
| | | | | | Prkcb, Kcnj5, Glra3, |
| | | | | | Gabrb1, Kcnj16, Gnb4, |
| | | | | | Prkcg, Kcnj6, Cacng4, |
| | | | | | Gabrb2, Grin2b, Lrrc7, |
| | | | | | Chrna7, Kcnj10, Chrna4, |
| | | | | | Chrnb3, Kcnj12, Chrna6 |
| R- | Ephrin signaling | −2.03 | 0.0383 | 0.29 | Src, Git1, Efnb2, Ephb6, |
| MMU- | | | | | Ephb1, Ephb2 |
| 3928664 | | | | | |
| R- | Neuronal System | −2.025 | 0 | 0.45 | Nefl, Lin7c, Kcna1, Ntrk3, |
| MMU- | | | | | Panx2, Gng3, Gabra1, |
| 112316 | | | | | Gabbr1, Prkaca, Gria4, |
| | | | | | Nlgn2, Gnal, Adcy5, Kcnj9, |
| | | | | | Chrna5, Camk2d, Kcnj15, |
| | | | | | Kcnh3, Syn1, Lrfn4, |
| | | | | | Kcnmb2, Gng2, Dlgap2, |
| | | | | | Slitrk5, Kcnk10, Prkar1b, |
| | | | | | Nrxn1, Prkacb, Lrrtm2, |
| | | | | | Kcnc2, Kcna2, Gabbr2, |
| | | | | | Grin2a, Grm1, Homer2, |
| | | | | | Arl6ip5, Dnajc5, Prkar2b, |
| | | | | | Lrrc4b, Gabrr2, Slc1a2, |
| | | | | | Gnb2, Adcy8, Lrfn3, Grik5, |
| | | | | | Kcnh1, Cacnb4, Syn2, |
| | | | | | Akap5, Kcnq5, Prkar1a, |
| | | | | | Grik4, Dlg4, Kcng1, |
| | | | | | Grin3a, Kcnd1, Epb4ll1, |
| | | | | | Ppfia3, Kcnh2, Cacng3, |
| | | | | | Stxbp1, Kcnf1, Kcna4, |
| | | | | | Lrfn2, Plcb1, Kcnv1, |
| | | | | | Dlgap1, Chrna3, Slitrk4, |
| | | | | | Chrnb4, Camkk2, Kcnd3, |
| | | | | | Rims1, Il1rap12, Ptprs, |
| | | | | | Kcnb1, Nsf, Cask, Kcnk9, |
| | | | | | Cacnala, Rps6ka2, Gabra3, |
| | | | | | Kcna6, Kcna3, Dlgap3, |
| | | | | | Kcna5, Grip1, Grm5, Dlg2, |
| | | | | | Ap2m1, Ap2a2, |
| | | | | | Kcnn3,Nlgn3, Kcnc4, |
| | | | | | Lrfn1, Calm3, Gng13, |
| | | | | | Camk2a, Slc17a7, Kcnh5, |
| | | | | | Cacnb1, Slitrk1, Cacnb3, |
| | | | | | Prkcb, Flot1, Kcnj14, |
| | | | | | Kcnj5, Nlgn1, Kcnh7, |
| | | | | | Glra3, Slc6a4, Hcn1, |
| | | | | | Cacna1b, Gabrb1, Shank2, |
| | | | | | Kcnj16, Shank1, Gnb4, |
| | | | | | Shank3, Prkcg, Kcnq4, |
| | | | | | Apba1, Kcnj6, Cacng4, |
| | | | | | Ptprf, Kcnma1, Gabrb2, |
| | | | | | Grin2b, Stx1a, Kcng3, |
| | | | | | Lrrc7, Kcnk2, Chrna7, |
| | | | | | Kcnj10, Kcnk1, Chrna4, |
| | | | | | Chrnb3, Kcnj12, Slc18a2, |
| | | | | | Chrna6, Slc6a3 |
| R- | Neurexins and | −1.973 | 0.0326 | 0.55 | Nlgn2, Dlgap2, Nrxn1, |
| MMU- | neuroligins | | | | Lrrtm2, Grm1, Homer2, |
| 6794361 | | | | | Dlg4, Epb4ll1, Dlgap1, |
| | | | | | Cask, Dlgap3, Grm5, Dlg2, |
| | | | | | Nlgn3, Nlgn1, Shank2, |
| | | | | | Shank 1, Shank3 |
| R- | COPI-independent | −1.957 | 0.0144 | 0.59 | Dync1i2, Dync1li1, Dctn1, |
| MMU- | Golgi-to-ER | | | | Tubb4b, Dync1li2, |
| 6811436 | retrograde traffic | | | | Dync1h1, Pafah1b1, |
| | | | | | Pafah1b2, Capzb, Rab6a, |
| | | | | | Capza2, Tuba1c, Dynll2, |
| | | | | | Bicd1, Dctn3, Agpat3, |
| | | | | | Dynl11, Dctn6, Tubb3, |
| | | | | | Tubb6, Tubb4a, Actr1a, |
| | | | | | Rab6b, Tubb2b, Tubb2a, |
| | | | | | Tuba1a, Tuba4a |
| R- | Voltage gated | −1.912 | 0.0232 | 0.51 | Kcnh3, Kcnc2, Kcna2, |
| MMU- | Potassium channels | | | | Kcnh1, Kcnq5, Kcng1, |
| 1296072 | | | | | Kcnd1, Kcnh2, Kcnf1, |
| | | | | | Kcna4, Kcnv1, Kcnd3, |
| | | | | | Kcnb1, Kcna6, Kcna3, |
| | | | | | Kcna5, Kcnc4, Kcnh5, |
| | | | | | Kcnh7, Kcnq4, Kcng3 |
| R- | Potassium Channels | −1.854 | 0.0074 | 0.47 | Gng3, Gabbr1, Kcnj9, |
| MMU- | | | | | Kcnj15, Kcnh3, Kcnmb2, |
| 1296071 | | | | | Gng2, Kcnk10, Kcnc2, |
| | | | | | Kcna2, Gabbr2, Gnb2, |
| | | | | | Kcnh1, Kcnq5, Kcng1, |
| | | | | | Kcnd1, Kcnh2, Kcnf1, |
| | | | | | Kcna4, Kcnv1, Kcnd3, |
| | | | | | Kcnb1, Kcnk9, Kcna6, |
| | | | | | Kcna3, Kcna5, Kcnn3, |
| | | | | | Kcnc4, Gng13, Kcnh5, |
| | | | | | Kcnj14, Kcnj5, Kcnh7, |
| | | | | | Hcn1, Kcnj16, Gnb4, |
| | | | | | Kcnq4, Kcnj6, Kcnma1, |
| | | | | | Kcng3, Kcnk2, Kcnj10, |
| | | | | | Kcnk1, Kcnj12 |
| R- | Transmission | −1.846 | 0.0001 | 0.4 | Gng3, Gabra1, Gabbr1, |
| MMU- | across Chemical | | | | Prkaca, Gria4, Gnal, |
| 112315 | Synapses | | | | Adcy5, Kcnj9, Chrna5, |
| | | | | | Camk2d, Kcnj15, Syn1, |
| | | | | | Gng2, Prkar1b, Prkacb, |
| | | | | | Gabbr2, Grin2a, Arl6ip5, |
| | | | | | Dnajc5, Prkar2b, Gabrr2, |
| | | | | | Slc1a2, Gnb2, Adcy8, |
| | | | | | Grik5, Cacnb4, Syn2, |
| | | | | | Akap5, Prkarla, Grik4, |
| | | | | | Dlg4, Grin3a, Epb4ll1, |
| | | | | | Ppfia3, Cacng3, Stxbp1, |
| | | | | | Plcb1, Chrna3, Chrnb4, |
| | | | | | Camkk2, Rims1, Nsf, Cask, |
| | | | | | Cacnala, Rps6ka2, Gabra3, |
| | | | | | Grip1, Dlg2, Ap2m4, |
| | | | | | Ap2a2, Calm3, Gng13, |
| | | | | | Camk2a, Slc17a7, Cacnb1, |
| | | | | | Cacnb3, Prkcb, Kcnj5, |
| | | | | | Glra3, Slc6a4, Cacna1b, |
| | | | | | Gabrb1, Kcnj16, Gnb4, |
| | | | | | Prkcg, Apbal, Kcnj6, |
| | | | | | Cacng4, Gabrb2, Grin2b, |
| | | | | | Stx1a, Lrrc7, Chrna7, |
| | | | | | Kcnj10, Chrna4, Chrnb3, |
| | | | | | Kcnj12, Slc18a2, Chrna6, |
| | | | | | Slc6a3 |
| R- | Transcriptional | −1.748 | 0.0365 | 0.34 | Psmd7, Trp53, Psmb5, |
| MMU- | regulation by | | | | Rps27a, Psmc2, Smad3, |
| 8878159 | RUNX3 | | | | Psmd2, Snw1, Ubc, Psmc5, |
| | | | | | Lef1, Yap1, Tcf712, Tcf7l1, |
| | | | | | Maml2, Psmb2, Ctnnb1, |
| | | | | | Psma6, Mamld1, Src, |
| | | | | | Hdac4, Cond1, Psmf1, |
| | | | | | Psma7, Tead3, Psmd8, |
| | | | | | Psmb10 |
| R- | Cardiac conduction | −1.669 | 0.0461 | 0.43 | Atp2a2, Camk2d, Atp2b1, |
| MMU- | | | | | Asph, Kcnk10, Kcnk5, |
| 5576891 | | | | | Cacng7, Stim1, Kcnip2, |
| | | | | | Atpla2, Fkbp1b, Atpla3, |
| | | | | | Kcnd1, Itpr1, Kcnh2, |
| | | | | | Atpla1, Ces1d, Kcnd3, |
| | | | | | Cacng6, Kcne11, Slc8a3, |
| | | | | | Kcnk9, Kcna5, Calm3, |
| | | | | | Camk2a, Akap9, Cacnb1, |
| | | | | | Fxyd2, Kcnj14, Atp2a3, |
| | | | | | Corin, Slc8a2, Fxyd7, |
| | | | | | Atp2b4, Fxyd4, Cacng4, |
| | | | | | Atp2b2, Pln, Kcne4, |
| | | | | | Kcnk2, Kcnip3, Kcnk1, |
| | | | | | Kcnj12 |
| R- | Muscle contraction | −1.646 | 0.0074 | 0.29 | Fkbp1b, Atp1a3, Kcnd1, |
| MMU- | | | | | Acta1, Itpr1, Kcnh2, |
| 397014 | | | | | Atp1a1, Ces1d, Kcnd3, |
| | | | | | Cacng6, Kcne11, Slc8a3, |
| | | | | | Kcnk9, Kcna5, Calm3, |
| | | | | | Camk2a, Neb, Akap9, |
| | | | | | Cacnb1, Mylpf, Fxyd2, |
| | | | | | Kcnj14, Atp2a3, Tnnc1, |
| | | | | | Corin, Slc8a2, Fxyd7, |
| | | | | | Atp2b4, Fxyd4, Tnnt1, |
| | | | | | Cacng4, Atp2b2, Pln, |
| | | | | | Kcne4, Lmod1, Kcnk2, |
| | | | | | Kcnip3, Actn3, Gucy1a2, |
| | | | | | Myh3, Kcnk1, Tmod4, |
| | | | | | Kcnj12 |
| R- | Neddylation | −1.571 | 0.0058 | 0.33 | Fbxo4, Vhl, Keap1, Ercc8, |
| MMU- | | | | | Dcun1d3, Klhl22, Asb8, |
| 8951664 | | | | | Rbx1, Dcun1d4, Psmd7, |
| | | | | | Fbxl14, Fem1b, Commd1, |
| | | | | | Psmb5, Asb13, Fbxo21, |
| | | | | | Ube2d2a, Fem1c, Vcp, |
| | | | | | Rps27a, Psmc2, Psmb11, |
| | | | | | Uch13, Fbx06, Klhl2, |
| | | | | | Fbx14, Asb11, Dcaf8, |
| | | | | | Fbxo31, Fbx18, Psmd2, |
| | | | | | Spsb3, Ubc, Psmc5, |
| | | | | | Dcun1d1, Dtl, Cops4, |
| | | | | | Dcaf7, Fbxo30, Cul4a, |
| | | | | | Nploc4, Dcaf5, Klhl5, |
| | | | | | Psmb2, Fbxl16, Socs5, |
| | | | | | Ube2m, Hif1a, Psma6, |
| | | | | | Fbxo10, Cul2, Wsb2, Gan, |
| | | | | | Fbxo22, Asb4, Commd5, |
| | | | | | Psmf1, Fbxw7, Fbxo41, |
| | | | | | Psma7, Asb5, Tulp4, |
| | | | | | Fbxo40, Cop1, Asb18, |
| | | | | | Psmd8, Fbxl21, Klhl11, |
| | | | | | Ankrd9, Fbxo17, Psmb10, |
| | | | | | Fbx032, Asb6, Asb14 |
| R- | Antigen processing: | −1.55 | 0.0032 | 0.39 | Ltn1, Fbx115, Cdc34, |
| MMU- | Ubiquitination and | | | | Rchy1, Npepps, Fbxo11, |
| 983168 | Proteasome | | | | Psmb9, Lrsam1, Herc3, |
| | degradation | | | | Fbx112, Ubr1, Ube2g1, |
| | | | | | Uba3, Psmd12, Fbxo2, |
| | | | | | Psme3, Klhl13, Rnf34, |
| | | | | | Tpp2, Pja2, Rnf14, Ube4a, |
| | | | | | Anapc4, Fzr1, Anapc2, |
| | | | | | Fbxo4, Vhl, Keap1, Ubox5, |
| | | | | | Klhl22, Asb8, Rbx1, |
| | | | | | Ube3b, Uba1, Rbck1, |
| | | | | | Cdc20, Psmd7, Fbxl14, |
| | | | | | Cdc27, Trim71, Trim37, |
| | | | | | Psmb5, Asb13, Fbxo21, |
| | | | | | Ubr2, Ube2d2a, Ube2k, |
| | | | | | Glmn, Rps27a, Psmc2, |
| | | | | | Psmb11, Rnf182, Unk1, |
| | | | | | Ube2r2, Fbx06, Ube2a, |
| | | | | | Klhl2, Fbxl4, Asb11, |
| | | | | | Fbxo31, Fbx18, Psmd2, |
| | | | | | Ubc, Psmc5, Fbxo30, Cblb, |
| | | | | | Mex3c, Huwe1, Ube20, |
| | | | | | Cul7, Klhl5, Ubr4, Psmb2, |
| | | | | | Fbxl16, Trim32, Znrf1, |
| | | | | | Ube2m, Psma6, Lnpep, |
| | | | | | Fbxo10, Mib2, Atg7, |
| | | | | | Herc2, Rnf123, Trim41, |
| | | | | | Cul2, Znrf2, Gan, Fbxo22, |
| | | | | | Asb4, Psmfl, Fbxw7, |
| | | | | | Fbxo41, Psma7, Sh3rf1, |
| | | | | | Anapc 7, Asb5, Rnf126, |
| | | | | | Stub1, Fbxo40, Asb18, |
| | | | | | Psmd8, Fbxl21, Klhl11, |
| | | | | | Thop1, Fbxo17, Psmb10, |
| | | | | | Fbxo32, Asb6, Asb14 |
| R- | Signaling by WNT | −1.416 | 0.0468 | 0.24 | Wnt7a, Gnao1, Ppp2r1a, |
| MMU- | | | | | Usp34, Psmd2, Tle1, Dvl2, |
| 195721 | | | | | Ubc, Kremen1, Psmc5, |
| | | | | | Lef1, Vps26a, Axin2, |
| | | | | | Tnks2, Plcb1, Tcf7l2, |
| | | | | | Ppp2cb, Wnt6, Tcf7l1, |
| | | | | | Psmb2, Cby1, Bc19, Leo1, |
| | | | | | Wnt2, Rspo2, Ctnnb1, |
| | | | | | Psma6, Ap2m1, Ap2a2, |
| | | | | | Ywhaz, Amer1, Men1, |
| | | | | | Calm3, Gng13, Camk2a, |
| | | | | | Kremen2, Rspo1, Prkcb, |
| | | | | | Psmf1, Wnt2b, Wnt9a, |
| | | | | | Psma7, Gnb4, Csnk2b, |
| | | | | | Prkcg, Psmd8, Sox7, Dkk2, |
| | | | | | Fzd8, Wnt11, Wnt7b, Fzd3, |
| | | | | | Psmb10, Wnt4 |
| R- | Neutrophil | 1.588 | 0.0008 | 0.23 | Lyz2, S100a8, Hbb-bs, |
| MMU- | degranulation | | | | Cyba, C5ar1, Ltf, Hbb-bt, |
| 6798695 | | | | | S100a9, Lcn2, Camp, Ppbp, |
| | | | | | Stbd1, H2-Q2, Tnfaip6, |
| | | | | | Itgb2, Hexb, Dsp, Plac8, |
| | | | | | Cybb, Ctss, Mmp25, |
| | | | | | Rab37, Ptpn6, Cd53, Ggh, |
| | | | | | Ptafr, Mmp8, Sell, Ptprc, |
| | | | | | B4galt1, Adam8, Ear2, |
| | | | | | Galns, Arhgap45, Chil1, |
| | | | | | Unc13d, Sting1, Acpp, |
| | | | | | Ifi204, Mpo, Cd177, |
| | | | | | Atp8b4, Frk, Bst1, Iqgap1, |
| | | | | | Ctsc, Tcirg1, Tbc1d10c, |
| | | | | | Prg2, S100a11, Tnfrsf1b, |
| | | | | | Gsdmd, Hp, C3ar1, Prcp, |
| | | | | | Mmp9, Abca13, Slc2a5, |
| | | | | | Slc27a2, Tlr2, Stk11ip, |
| | | | | | Rnaset2a, Elane, Cd33, |
| | | | | | Qpct, Cxcr2, Fcgr4, Dock2, |
| | | | | | Alox5, Plaur, Arpc5, H2- |
| | | | | | Q10, Cfp, Atp6v0c, Mgst1, |
| | | | | | Nckap11, Creg1, Anxa2, |
| | | | | | Dpp7, Mgam, Mvp, |
| | | | | | Aldh3b1, Aga, Dynlt1f, |
| | | | | | Cst3, B2m, Dsgla, Ist1, |
| | | | | | Mme, Cd36, Ticam2, |
| | | | | | Lamtor2, Surf4, Lilra5, |
| | | | | | Clec5a, Cd68, H2-Q4, |
| | | | | | Serpinb1a, Anpep, Rab31, |
| | | | | | Prtn3, Atad3a, Pgam1, H2- |
| | | | | | Q1, Stk 10, Vcl |
| R- | Immunoregulatory | 1.706 | 0.0354 | 0.41 | H2-Q2, Itgb2, Cd22, |
| MMU- | interactions | | | | Col1a2, Sell, Pvr, Itgb7, |
| 198933 | between a | | | | Col3a1, Col1a1, Col17a1, |
| | Lymphoid and a | | | | Icam2, Cd200r2, Cd300lg, |
| | non-Lymphoid cell | | | | Siglec1, Cd33, Fcgr4, |
| | | | | | Col2a1, Cd34, H2-Q10, |
| | | | | | Trem1, Pilra, Crtam, B2m, |
| | | | | | H2-Q4, H2-Q1, Treml1, |
| | | | | | Cd300c2, Cd226, Fcgr2b, |
| | | | | | Cd1d1, H2-T23, Lair1, |
| | | | | | Host, Siglece |
| R- | Cell surface | 1.736 | 0.0158 | 0.3 | Jchain, Cd48, Cd74, Procr, |
| MMU- | interactions at the | | | | Itgb2, Mertk, Thbd, Ptpn6, |
| 202733 | vascular wall | | | | Col1a2, Sell, Spn, Col1a1, |
| | | | | | Jam3, Inpp5d, Lyn, Fn1, |
| | | | | | Sdc4, Cd177, Esam, |
| | | | | | Angpt2, Sele, Trem1, |
| | | | | | Apob, Sdc1, Jam2, Pros1 |
| R- | Extracellular matrix | 1.79 | 0.0003 | 0.42 | Tnn, P3h2, Pcolce, Col6a1, |
| MMU- | organization | | | | Itgb2, Col6a2, Col24a1, |
| 1474244 | | | | | Tmprss6, Ctss, Mmp25, |
| | | | | | Ibsp, Colgalt1, Col11a1, |
| | | | | | Mmp8, Col1a2, Bgn, |
| | | | | | Col8al, Col26al, Pcolce2, |
| | | | | | Adam8, Itgb7, Col3a1, |
| | | | | | Col23a1, Col1a1, Col4a5, |
| | | | | | Col7a1, Col17a1, Jam3, |
| | | | | | Icam2, Fn1, Fbln2, Bmp1, |
| | | | | | Col8a2, Cdh1, Sdc4, Itga8, |
| | | | | | Crtap, Mmp9, Col4a4, |
| | | | | | Mfap5, Col6a5, Fbn1, |
| | | | | | Elane, Col4a1, Serpinh1, |
| | | | | | P4ha1, Matn3, Nid1, |
| | | | | | Capn3, Capn15, Col2a1, |
| | | | | | Col5a3, Mmp12, Fbln5, |
| | | | | | Vwf, Spp1, Htra1, Col4a2, |
| | | | | | Plg, Sdc1, Col14a1, |
| | | | | | Col5al, Jam2, Mmp11, |
| | | | | | Pxdn, Col9a3, Col16a1, |
| | | | | | Acan, A2m, Col4a6, Ltbp2, |
| | | | | | Mmp13, Matn4, P3h1, |
| | | | | | Col11a2, Loxl1, P4ha2, |
| | | | | | Capn8, Col19al, Itga9, |
| | | | | | Capn5, Klkb1, F11r, Itga2, |
| | | | | | Mfap4, Col12a1, Lox, Dcn, |
| | | | | | Mmp19, Vtn, Ddr2, Prss3, |
| | | | | | Emilin2 |
| R- | NCAM1 | 1.823 | 0.0429 | 0.56 | Col6a1, Col6a2, Col3a1, |
| MMU- | interactions | | | | Col6a5, Col4a1, Col2a1, |
| 419037 | | | | | Col5a3, Col4a2, Col5a1, |
| | | | | | Col9a3 |
| R- | GPVI-mediated | 1.832 | 0.0383 | 0.38 | Ptpn6, Rac2, Col1a2, Vav1, |
| MMU- | activation cascade | | | | Col1a1, Pik3r5, Lyn, Lcp2, |
| 114604 | | | | | Clec1b, Pik3r6, Lat, Plcg2, |
| | | | | | Syk, Pik3cg |
| R- | RHO GTPases | 1.842 | 0.0468 | 0.26 | S100a8, Cyba, S100a9, |
| MMU- | Activate NADPH | | | | Cybb, Rac2, Ncf1, Nox3 |
| 5668599 | Oxidases | | | | |
| R- | Keratan sulfate | 1.893 | 0.0203 | 0.42 | Ogn, Omd, Hexb, Fmod, |
| MMU- | degradation | | | | Prelp, Galns |
| 2022857 | | | | | |
| R- | Antimicrobial | 1.959 | 0.0068 | 0.27 | Lyz2, S100a8, Ltf, S100a9, |
| MMU- | peptides | | | | Lcn2, Camp, Rnase6, |
| 6803157 | | | | | Atp7a, Tlr2, Elane, Tlr1 |
| R- | Regulation of TLR | 1.963 | 0.0074 | 0.56 | S100a8, S100a9, Tlr4, |
| MMU- | by endogenous | | | | Gsdmd, Tlr2, Tlr6, Gsdme, |
| 5686938 | ligand | | | | Tlr1, Apob, Cd36 |
| R- | Degradation of the | 1.968 | 0.0003 | 0.48 | Col6a1, Col6a2, Tmprss6, |
| MMU- | extracellular matrix | | | | Ctss, Mmp25, Col11a1, |
| 1474228 | | | | | Mmp8, Col1a2, Col8a1, |
| | | | | | Col26a1, Adam8, Col3a1, |
| | | | | | Col1a1, Col4a5, Col7a1, |
| | | | | | Fn1, Col8a2, Cdh1, Mmp9, |
| | | | | | Col6a5, Fbn1, Elane, |
| | | | | | Col4a1, Nid1, Capn3, |
| | | | | | Capn15, Col2a1, Col5a3, |
| | | | | | Mmp12, Spp1, Htral, |
| | | | | | Col4a2, Plg, Col5a1, |
| | | | | | Mmp11, Acan, A2m, |
| | | | | | Col4a6, Mmp13, Col11a2, |
| | | | | | Capn8, Col19a1, Capn5, |
| | | | | | Klkb1, Col12a1, Den, |
| | | | | | Mmp19 |
| R- | Integrin cell surface | 1.99 | 0.001 | 0.48 | Col6a1, Itgb2, Col6a2, |
| MMU- | interactions | | | | Ibsp, Col1a2, Col8a1, |
| 216083 | | | | | Itgb7, Col3a1, Col1a1, |
| | | | | | Col4a5, Col7al, Jam3, |
| | | | | | Icam2, Fn1, Col8a2, Cdh1, |
| | | | | | Itga8, Col4a4, Col6a5, |
| | | | | | Fbn1, Col4a1, Col2a1, |
| | | | | | Col5a3, Vwf, Spp1, |
| | | | | | Col4a2, Col5a1, Jam2, |
| | | | | | Col9a3, Col16a1, Col4a6 |
| R- | Collagen | 2.005 | 0.0028 | 0.51 | Col6a1, Col6a2, Tmprss6, |
| MMU- | degradation | | | | Col11a1, Mmp8, Col1a2, |
| 1442490 | | | | | Col8a1, Col26a1, Col3a1, |
| | | | | | Col1a1, Col4a5, Col7a1, |
| | | | | | Col8a2, Mmp9, Col6a5, |
| | | | | | Col4a1, Col2a1, Col5a3, |
| | | | | | Mmp12, Col4a2, Col5a1, |
| | | | | | Mmp11, Col4a6, Mmp13, |
| | | | | | Col11a2 |
| R- | Collagen chain | 2.098 | 0.0006 | 0.54 | Col6a1, Col6a2, Col24a1, |
| MMU- | trimerization | | | | Col11al, Col1a2, Col8a1, |
| 8948216 | | | | | Col26al, Col3a1, Col23a1, |
| | | | | | Col1a1, Col7a1, Col17a1, |
| | | | | | Col8a2, Col6a5, Col2a1, |
| | | | | | Col5a3, Col14a1, Col5a1, |
| | | | | | Col9a3, Col16a1 |
| R- | Collagen | 2.145 | 0.0001 | 0.58 | P3h2, Pcolce, Col6a1, |
| MMU- | biosynthesis and | | | | Col6a2, Col24a1, Colgalt1, |
| 1650814 | modifying enzymes | | | | Col11a1, Col1a2, Col8a1, |
| | | | | | Col26a1, Pcolce2, Col3a1, |
| | | | | | Col23a1, Col1a1, Col4a5, |
| | | | | | Col7a1, Col17a1, Bmp1, |
| | | | | | Col8a2, Crtap, Col4a4, |
| | | | | | Col6a5, Col4a1, Serpinh1, |
| | | | | | P4ha1, Col2a1, Col5a3, |
| | | | | | Col4a2, Col14a1, Col5a1, |
| | | | | | Col9a3, Col16a1, Col4a6, |
| | | | | | P3h1, Col11a2, P4ha2 |
| R- | Collagen formation | 2.185 | 0 | 0.56 | P3h2, Pcolce, Col6a1, |
| MMU- | | | | | Col6a2, Col24a1, Ctss, |
| 1474290 | | | | | Colgalt1, Col11a1, Col1a2, |
| | | | | | Col8a1, Col26al, Pcolce2, |
| | | | | | Col3a1, Col23a1, Col1a1, |
| | | | | | Col4a5, Col7a1, Col17a1, |
| | | | | | Bmp1, Col8a2, Crtap, |
| | | | | | Mmp9, Col4a4, Col6a5, |
| | | | | | Col4a1, Serpinh1, P4ha1, |
| | | | | | Col2a1, Col5a3, Col4a2, |
| | | | | | Col14a1, Col5a1, Pxdn, |
| | | | | | Col9a3, Col16a1, Col4a6, |
| | | | | | Mmp13, P3h1, Col11a2, |
| | | | | | Loxl1, P4ha2 |
| R- | Assembly of | 2.261 | 0 | 0.63 | Pcolce, Col6a1, Col6a2, |
| MMU- | collagen fibrils and | | | | Col24a1, Ctss, Col11a1, |
| 2022090 | other multimeric | | | | Col1a2, Col8a1, Col3a1, |
| | structures | | | | Col1a1, Col4a5, Col7a1, |
| | | | | | Bmp1, Col8a2, Mmp9, |
| | | | | | Col6a5, Col4a1, Col2a1, |
| | | | | | Col5a3, Col4a2, Col14a1, |
| | | | | | Col5a1, Pxdn, Col9a3, |
| | | | | | Col4a6, Mmp13, Col11a2, |
| | | | | | Loxl1 |

Pathways significantly enriched (fdr corrected pvalue < 0.05) in either condition shown.
Positive normalized enrichment score greater than 1 signifies an increase with 5D supplementation.
Core genes implicated in each pathway shown in the last column.

### Other Embodiments

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

We claim:

1. A method for treating autism spectrum disorder in a subject, comprising administering to the subject:
(i) a composition comprising a therapeutically effective amount of one or more metabolites selected from glutamate, malate, ursodeoxycholate, 5-dodecenoate, N-acetyl-L-glutamate, citrate, glycodeoxycholate, or carboxyethyl aminobutyric acid (CEGABA); or
(ii) a composition comprising one or more bacterial species selected from Bifidobacterium bifidum, Eggerthella lento, Eisenbergiella massilien, Prevotella copri, Romboutsia timonensis, Blautia wexlerae, Ruminiclostridium siraeum, Bacteroides intestinalis, Faecalicatena lactaris, Dialister invisus, or Ruminococcus callidus.

2. The method of claim 1, wherein the composition comprises (i) two, three, four, or more of the metabolites; or (ii) two, three, four, or more of the bacterial species.

3. The method of claim 1, wherein the composition comprises a therapeutically effective amount of two or more metabolites selected from glutamate, malate, ursodeoxycholate, 5-dodecenoate, N-acetyl-L-glutamate, citrate, glycodeoxycholate, and carboxyethyl aminobutyric acid (CEGABA).

4. The method of claim 1, further comprising detecting a dysbiosis associated with autism spectrum disorder in a sample from the subject.

5. The method of claim 4, wherein the sample is a fecal sample.

6. The method of claim 4, wherein detecting the dysbiosis associated with autism spectrum disorder comprises:
(i) determining bacterial gene expression in the sample from the subject;
(ii) determining bacterial composition in the sample from the subject; or
(iii) both (i) and (ii).

7. The method of claim 4, wherein detecting the dysbiosis associated with autism spectrum disorder comprises determining that a bacterial species from the Akkermansiaceae family, Lachnospiraceae family, Streptococcaceae family, Pasteurellaceae family, Ruminococcaceae family, Bacteroidaceae family, Butyricicoccaceae family, Streptococcus genus, Blautia genus, Haemophilus genus, Faecalibacterium genus, Bacteroides genus, Roseburia genus, Fusicatenibacter genus, Lachnospira genus, Agathobaculum genus, or a combination thereof is depleted in the sample from the subject.

8. The method of claim 7, wherein the bacterial species that is depleted in the sample from the subject is selected from Blautia wexlerae, Bacteroides vulgatus, Bacteroides ovatus, Roseburia inulinivorans, Roseburia intestinalis, Fusicatenibacter saccharivorans, or Agathobaculum butyriciproducens.

9. The method of claim 8, wherein the bacterial species that is enriched in the sample from the subject is selected from Bacteroides thetaiotaomicron, Borfalki ceftriaxensis, and Faecalicatena torques.

10. The method of claim 4, wherein detecting the dysbiosis associated with autism spectrum disorder comprises determining that a bacterial species from the Bacteroidaceae family, Lachnospiraceae family, Oscillospiraceae family, Anaerovoraceae family, Erysipelotrichaceae family, Christensenellaceae family, Bacteroides genus, Blautia genus, Holdemania genus, Borkfalki genus, Anaerotignum genus, Faecalicatena genus, or a combination thereof is enriched in the sample from subject.

11. The method of claim 1, wherein the subject has severe autism identified using Mobile Autism Risk Assessment (MARA).

12. The method of claim 1, wherein the method comprises administering the composition to the subject once, twice, or three times per day, using an oral administration form selected from a tablet, a capsule, a powder, or a liquid.

13. The method of claim 1, further comprising administering another treatment for autism spectrum disorder to the subject.

14. The method of claim 1, wherein the subject is a human previously identified as having autism spectrum disorder prior to administering the composition.

15. A method of treating autism spectrum disorder or modulating anxiety in a subject, the method comprising administering to the subject a composition comprising one or more bacterial species of a bacterial family selected from Streptococcaceae, Lachnospiraceae, Ruminococcaceae, Bacteroidaceae, Butyricicoccaceae, and Pasteurellaceae.

16. The method of claim 15, wherein the one or more bacterial species are of a bacterial genera selected from Streptococcus, Blautia, Haemophilus, Faecalibacterium, Bacteroides, Roseburia, Fusicatenibacter, Lachnospira, or Agathobaculum.

17. The method of claim 15, wherein the one or more bacterial species are selected from Blautia wexlerae, Bacteroides vulgatus, Bacteroides ovatus, Roseburia inulinivorans, Roseburia intestinalis, Fusicatenibacter saccharivorans, and Agathobaculum butyriciproducens.

18. The method of claim 15, wherein the one or more bacterial species have a 16S rRNA selected from SEQ ID NOs 1-13.

19. A composition comprising:
(i) a therapeutically effective amount of one or more metabolites selected from glutamate, malate, ursodeoxycholate, 5-dodecenoate, N-acetyl-L-glutamate, citrate, glycodeoxycholate, or carboxyethyl aminobutyric acid (CEGABA);
(ii) one or more bacterial species selected from Bifidobacterium bifidum, Eggerthella lenta, Eisenbergiella massilien, Prevotella copri, Romboutsia timonensis, Blautia wexlerae, Ruminiclostridium siraeum, Bacteroides intestinalis, Faecalicatena lactaris, Dialister invisus, or Ruminococcus callidus; or
(iii) one or more bacterial species of a bacterial family selected from Streptococcaceae, Lachnospiraceae, Ruminococcaceae, Bacteroidaceae, Butyricicoccaceae, or Pasteurellaceae.

20. The composition of claim 19, wherein the composition comprises the one or more bacterial species of the bacterial family selected from Streptococcaceae, Lachnospiraceae, Ruminococcaceae, Bacteroidaceae, Butyricicoccaceae, and Pasteurellaceae and the one or more bacterial species are of a bacterial genera selected from Streptococcus, Blautia, Haemophilus, Faecalibacterium, Bacteroides, Roseburia, Fusicatenibacter, Lachnospira, or Agathobaculum.

21. The composition of claim 20, wherein the one or more bacterial species are selected from Blautia wexlerae, Bacteroides vulgatus, Bacteroides ovatus, Roseburia inulinivorans, Roseburia intestinalis, Fusicatenibacter saccharivorans, and Agathobaculum butyriciproducens.

22. The composition of claim 19, comprising:
(i) a therapeutically effective amount of two or more metabolites selected from glutamate, malate, ursodeoxycholate, 5-dodecenoate, N-acetyl-L-glutamate, citrate, glycodeoxycholate, and carboxyethyl aminobutyric acid (CEGABA);
(ii) two or more bacterial species selected from Bifidobacterium bifidum, Eggerthella lenta, Eisenbergiella massilien, Prevotella copri, Romboutsia timonensis, Blautia wexlerae, Ruminiclostridium siraeum, Bacteroides intestinalis, Faecalicatena lactaris, Dialister invisus, and Ruminococcus callidus; or
(iii) two or more bacterial species of a bacterial family selected from Streptococcaceae, Lachnospiraceae, Ruminococcaceae, Bacteroidaceae, Butyricicoccaceae, and Pasteurellaceae.

23. The composition of claim 19, wherein the composition is formulated for oral administration, optionally as a tablet, a capsule, a powder, or a liquid.


SEQUENCE LISTING (15 sequences)

SEQ ID NO: 1 - DNA, 233 bp, Faecalicatena lactaris
  source 1..233: mol_type = unassigned DNA
gcaagcgttgtccggaattactgggtgtaaagggagcgcaggcggatttgcaagttggaagtgaaacccatgggctcaacccatggactgctttcaaaactgcagatcttgagtggtgtagaggtaggcggaattcccggtgtagcggtggaatgcgtagatatcgggaggaacaccagtggcgaaggcggcctactgggcactaactgacgctgaggctcgaaagcatgggt

SEQ ID NO: 2 - DNA, 233 bp, Dialister invisus
  source 1..233: mol_type = unassigned DNA
gcaagcgttatccggatttactgggtgtaaagggagcgtagacggacgggcaagtctgatgtgaaaggcaggggctcaacccctggactgcattggaaactgttcatcttgagtgccggagaggtaagcggaattcctagtgtagcggtgaaatgcgtagatattaggaggaacaccagtggcgaaggcggcttactggacggtaactgacgttgaggctcgaaagcgtgggg

SEQ ID NO: 3 - DNA, 233 bp, Faecalibacterium spp.
  source 1..233: mol_type = unassigned DNA
acaagcgttgtccggaattactgggtgtaaagggagcgcaggcgggagaacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggt

SEQ ID NO: 4 - DNA, 233 bp, Bacteroides vulgatus
  source 1..233: mol_type = unassigned DNA
ccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggt

SEQ ID NO: 5 - DNA, 233 bp, Bacteroides ovatus; identical to SEQ ID NO: 4
  source 1..233: mol_type = unassigned DNA

SEQ ID NO: 6 - DNA, 233 bp, Roseburia inulinivorans
  source 1..233: mol_type = unassigned DNA
gcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtgggg

SEQ ID NO: 7 - DNA, 233 bp, Roseburia intestinalis
  source 1..233: mol_type = unassigned DNA
gcaagcgttatccggatttactgggtgtaaagggagcgcaggcggtacggcaagtctgatgtgaaagcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtgggg

SEQ ID NO: 8 - DNA, 233 bp, Faecalicatena torques
  source 1..233: mol_type = unassigned DNA
gcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtgggg

SEQ ID NO: 9 - DNA, 233 bp, Fusicatenibacter saccharivorans
  source 1..233: mol_type = unassigned DNA
gcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtgggg

SEQ ID NO: 10 - DNA, 233 bp, Lachnospira spp.
  source 1..233: mol_type = unassigned DNA
gcaagcgttatccggatttactgggtgtaaagggagtgtaggtggccatgcaagtcagaagtgaaaatccggggctcaaccccggaactgcttttgaaactgtaaggctagagtgcaggaggggtgagtggaattcctagtgtagcggtgaaatgcgtagatattaggaggaacaccagtggcgaaggcggctcactggactgtaactgacactgaggctcgaaagcgtgggg

SEQ ID NO: 11 - DNA, 233 bp, Lachnospiraceae sp.
  source 1..233: mol_type = unassigned DNA
gcaagcgttatccggatttactgggtgtaaagggagtgtaggtggtatcacaagtcagaagtgaaagcccggggctcaaccccgggactgcttttgaaactgtggaactggagtgcaggagaggtaagtggaattcctagtgtagcggtgaaatgcgtagatattaggaggaacaccagtggcgaaggcggcttactggactgtaactgacactgaggctcgaaagcgtgggg

SEQ ID NO: 12 - DNA, 233 bp, Agathobaculum butyriciproducens
  source 1..233: mol_type = unassigned DNA
gcaagcgttatccggatttactgggtgtaaagggcgcgcaggcgggccggcaagttggaagtgaaatctatgggcttaacccataaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtgggg

SEQ ID NO: 13 - DNA, 233 bp, Agathobaculum butyriciproducens
  source 1..233: mol_type = unassigned DNA
gcaagcgttatccggatttactgggtgtaaagggcgcgcaggcgggccggtaagttggaagtgaaatctatgggcttaacccataaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtgggg

SEQ ID NO: 14 - DNA, 60 bp, synthetic construct
  source 1..60: mol_type = other DNA
aatgatacggcgaccaccgagatctacactatggtaattgtgtgycagcmgccgcggtaa

SEQ ID NO: 15 - DNA, 68 bp, synthetic construct
  source 1..68: mol_type = other DNA
caagcagaagacggcatacgagatnnnnnnnnnnnnagtcagtcagccggactacnvgggtwtctaat